(19) United States
(12) Patent Application Publication
(10) Pub. No.: US 2022/0152164 A1
(43) Pub. Date: May 19, 2022

(54) GPCR COMBINATION THERAPIES

(71) Applicant: Kallyope, Inc., New York, NY (US)

(72) Inventors: Nancy THORNBERRY, Westfield, NJ (US); Ann WEBER, Cranford, NJ (US); Maja ENGELSTOFT, New York, NY (US); Zac FARINO, New York, NY (US); Ian PEIKON, Brooklyn, NY (US); Shirly PINTO, Short Hills, NJ (US); Iyassu SEBHAT, Jersey City, NJ (US); Paul RICHARDS, New York, NY (US)

(21) Appl. No.: 17/440,901

(22) PCT Filed: Mar. 19, 2020
(86) PCT No.: PCT/US2020/023611

§ 371 Date: Sep. 20, 2021

Related U.S. Application Data
(60) Provisional application No. 62/851,510, filed on May 22, 2019.
(60) Provisional application No. 62/822,659, filed on Mar. 22, 2019.

(51) Int. Cl. A61K 38/48; A61K 45/06; A61P 3/04

(52) U.S. Cl. CPC A61K 38/4813; A61P 3/04; A61K 45/06

(57) ABSTRACT

This disclosure is directed, at least in part, to GPCR modulators in combination therapies useful for the treatment of conditions or disorders involving the gut-brain axis. In some embodiments, these modulators are gut-restricted compounds. In some embodiments, these modulators are GPCR agonists, antagonists, inverse agonists, neutral antagonists, positive allosteric modulators, or negative allosteric modulators. In some embodiments, the condition or disorder is a metabolic disorder, such as diabetes or obesity, or a nutritional disorder such as short bowel syndrome.

### CROSS-REFERENCE TO RELATED APPLICATIONS

This application claims the benefit of U.S. Provisional Application No. 62/822,659 filed on Mar. 22, 2019, and U.S. Provisional Application No. 62/851,510 filed on May 22, 2019, each of which is incorporated herein by reference in its entirety.

### BRIEF SUMMARY OF THE INVENTION

Disclosed herein, in certain embodiments, are G-Protein Coupled Receptor (GPCR) modulator combination therapies useful for the treatment of conditions or disorders involving the gut-brain axis. In some embodiments, the GPCR modulators are gut-restricted or selectively modulate GPCRs located in the gut. In some embodiments, the condition is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, celiac disease, and enteritis, including chemotherapy-induced enteritis or radiation-induced enteritis; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis.

In some embodiments, disclosed herein is method of treating a condition or disorder involving the gut-brain axis in an individual in need thereof, the method comprising administering to the individual at least two receptor modulators selected from the group consisting of a TGR5 receptor modulator, a GPR40 receptor modulator, a GPR119 receptor modulator, a somatostatin receptor 5 (SSTR5) modulator, and a CCK_{A} receptor modulator; wherein at least one of the group consisting of i) the TGR5 receptor modulator, ii) the GPR40 receptor modulator, iii) the GPR119 receptor modulator, iv) the somatostatin receptor 5 (SSTR5) modulator, and v) the CCK_{A} receptor modulator is a gut-restricted modulator.

In some embodiments, the method comprises administering to the individual: i) a TGR5 receptor modulator and a SSTR5 receptor modulator; ii) a TGR5 receptor modulator and a GPR40 receptor modulator; iii) a TGR5 receptor modulator and a GPR119 receptor modulator; iv) a TGR5 receptor modulator and a CCK_{A} receptor modulator; v) a GPR40 receptor modulator and a GPR119 receptor modulator; vi) a GPR40 receptor modulator and a SSTR5 receptor modulator; vii) a GPR40 receptor modulator and a CCK_{A} receptor modulator; viii) a GPR119 receptor modulator and a SSTR5 receptor modulator; ix) a GPR119 receptor modulator and a CCK_{A} receptor modulator; or x) a SSTR5 receptor modulator and a CCK_{A} receptor modulator. In some embodiments, the method comprises administering to the individual: i) a TGR5 receptor modulator, a SSTR5 receptor modulator, and a GPR119 receptor modulator; ii) a TGR5 receptor modulator, a SSTR5 receptor modulator, and a GPR40 receptor modulator; iii) a TGR5 receptor modulator, a SSTR5 receptor modulator, and a CCK_{A} receptor modulator; iv) a TGR5 receptor modulator, a GPR40 receptor modulator, and a GPR119 receptor modulator; v) a TGR5 receptor modulator, a GPR40 receptor modulator, and a CCK_{A} receptor modulator; vi) a TGR5 receptor modulator, a GPR119 receptor modulator, and a CCK_{A} receptor modulator; vii) a GPR40 receptor modulator, a GPR119 receptor modulator, and a SSTR5 receptor modulator; viii) a SSTR5 receptor modulator, a GPR40 receptor modulator, and a CCK_{A} receptor modulator; ix) a SSTR5 receptor modulator, a GPR119 receptor modulator, and a CCK_{A} receptor modulator; or x) a GPR40 receptor modulator, a GPR119 receptor modulator, and a CCK_{A} receptor modulator. In some embodiments, the method comprises administering to the individual: i) a TGR5 receptor modulator, a GPR40 receptor modulator, a GPR119 receptor modulator, and a SSTR5 receptor modulator; ii) a TGR5 receptor modulator, a GPR40 receptor modulator, a GPR119 receptor modulator, and a CCK_{A} receptor modulator; iii) a TGR5 receptor modulator, a GPR40 receptor modulator, a SSTR5 receptor modulator, and a CCK_{A} receptor modulator; iv) a TGR5 receptor modulator, a GPR119 receptor modulator, a SSTR5 receptor modulator, and a CCK_{A} receptor modulator; v) a GPR40 receptor modulator, a GPR119 receptor modulator, and a SSTR5 receptor modulator, and a CCK_{A} receptor modulator; or vi) a TGR5 receptor modulator, a GPR40 receptor modulator, a GPR119 receptor modulator, a SSTR5 receptor modulator, and a CCK_{A} receptor modulator.

In some embodiments, the method comprises administering to the individual: i) a TGR5 receptor modulator and a GPR40 receptor modulator; ii) a TGR5 receptor modulator and a GPR119 receptor modulator; or iii) a GPR40 receptor modulator and a GPR119 receptor modulator. In some embodiments, the method comprises administering to the individual a TGR5 receptor modulator, a GPR40 receptor modulator, and a GPR119 receptor modulator. In some embodiments, the method further comprises administering to the individual a SSTR5 receptor modulator. In some embodiments, the method further comprises administering to the individual a CCK_{A} receptor modulator.

In some embodiments, the method further comprises administering to the individual a PDE4 inhibitor. In some embodiments, the method comprises administering to the individual: i) a TGR5 receptor modulator, a SSTR5 receptor modulator, and a PDE4 inhibitor; ii) a TGR5 receptor modulator, a GPR40 receptor modulator, and a PDE4 inhibitor; iii) a TGR5 receptor modulator, a GPR119 receptor modulator, and a PDE4 inhibitor; iv) a TGR5 receptor modulator, a CCK_{A} receptor modulator, and a PDE4 inhibitor; v) a GPR40 receptor modulator, a GPR119 receptor modulator, and a PDE4 inhibitor; vi) a GPR40 receptor modulator, a SSTR5 receptor modulator, and a PDE4 inhibitor; vii) a GPR40 receptor modulator, a CCK_{A} receptor modulator, and a PDE4 inhibitor; viii) a GPR119 receptor modulator, a SSTR5 receptor modulator, and a PDE4 inhibitor; ix) a GPR119 receptor modulator, a CCK_{A} receptor modulator, and a PDE4 inhibitor; or x) a SSTR5 receptor modulator, a CCK_{A} receptor modulator, and a PDE4 inhibitor. In some embodiments, the method comprises administering to the individual: i) a TGR5 receptor modulator, a SSTR5 receptor modulator, a GPR119 receptor modulator, and a PDE4 inhibitor; ii) a TGR5 receptor modulator, a SSTR5 receptor modulator, a GPR40 receptor modulator, and a PDE4 inhibitor; iii) a TGR5 receptor modulator, a SSTR5 receptor modulator, a CCK_{A} receptor modulator, and a PDE4 inhibitor; iv) a TGR5 receptor modulator, a GPR40 receptor modulator, a GPR119 receptor modulator, and a PDE4 inhibitor; v) a TGR5 receptor modulator, a GPR40 receptor modulator, a CCK_{A} receptor modulator, and a PDE4 inhibitor; vi) a TGR5 receptor modulator, a GPR119 receptor modulator, a CCK_{A} receptor modulator, and a PDE4 inhibitor; vii) a GPR40 receptor modulator, a GPR119 receptor modulator, a SSTR5 receptor modulator, and a PDE4 inhibitor; viii) a SSTR5 receptor modulator, a GPR40 receptor modulator, a CCK_{A} receptor modulator, and a PDE4 inhibitor; ix) a SSTR5 receptor modulator, a GPR119 receptor modulator, a CCK_{A} receptor modulator, and a PDE4 inhibitor; or x) a GPR40 receptor modulator, a GPR119 receptor modulator, a CCK_{A} receptor modulator, and a PDE4 inhibitor. In some embodiments, the method comprises administering to the individual: i) a TGR5 receptor modulator, a GPR40 receptor modulator, a GPR119 receptor modulator, a SSTR5 receptor modulator, and a PDE4 inhibitor; ii) a TGR5 receptor modulator, a GPR40 receptor modulator, a GPR119 receptor modulator, a CCK_{A} receptor modulator, and a PDE4 inhibitor; iii) a TGR5 receptor modulator, a GPR40 receptor modulator, a SSTR5 receptor modulator, a CCK_{A} receptor modulator, and a PDE4 inhibitor; iv) a TGR5 receptor modulator, a GPR119 receptor modulator, a SSTR5 receptor modulator, a CCK_{A} receptor modulator, and a PDE4 inhibitor; v) a GPR40 receptor modulator, a GPR119 receptor modulator, a SSTR5 receptor modulator, a CCK_{A} receptor modulator, and a PDE4 inhibitor; or vi) a TGR5 receptor modulator, a GPR40 receptor modulator, a GPR119 receptor modulator, a SSTR5 receptor modulator, a CCK_{A} receptor modulator, and a PDE4 inhibitor.

In some embodiments, the method further comprises administering to the individual a DPP-4 inhibitor. In some embodiments, the method comprises administering to the individual: i) a TGR5 receptor modulator, a SSTR5 receptor modulator, and a DPP-4 inhibitor; ii) a TGR5 receptor modulator, a GPR40 receptor modulator, and a DPP-4 inhibitor; iii) a TGR5 receptor modulator, a GPR119 receptor modulator, and a DPP-4 inhibitor; iv) a TGR5 receptor modulator, a CCK_{A} receptor modulator, and a DPP-4 inhibitor; v) a GPR40 receptor modulator, a GPR119 receptor modulator, and a DPP-4 inhibitor; vi) a GPR40 receptor modulator, a SSTR5 receptor modulator, and a DPP-4 inhibitor; vii) a GPR40 receptor modulator, a CCK_{A} receptor modulator, and a DPP-4 inhibitor; viii) a GPR119 receptor modulator, a SSTR5 receptor modulator, and a DPP-4 inhibitor; ix) a GPR119 receptor modulator, a CCK_{A} receptor modulator, and a DPP-4 inhibitor; or x) a SSTR5 receptor modulator, a CCK_{A} receptor modulator, and a DPP-4 inhibitor. In some embodiments, the method comprises administering to the individual: i) a TGR5 receptor modulator, a SSTR5 receptor modulator, a GPR119 receptor modulator, and a DPP-4 inhibitor; ii) a TGR5 receptor modulator, a SSTR5 receptor modulator, a GPR40 receptor modulator, and a DPP-4 inhibitor; iii) a TGR5 receptor modulator, a SSTR5 receptor modulator, a CCK_{A} receptor modulator, and a DPP-4 inhibitor; iv) a TGR5 receptor modulator, a GPR40 receptor modulator, a GPR119 receptor modulator, and a DPP-4 inhibitor; v) a TGR5 receptor modulator, a GPR40 receptor modulator, a CCK_{A} receptor modulator, and a DPP-4 inhibitor; vi) a TGR5 receptor modulator, a GPR119 receptor modulator, a CCK_{A} receptor modulator, and a DPP-4 inhibitor; vii) a GPR40 receptor modulator, a GPR119 receptor modulator, a SSTR5 receptor modulator, and a DPP-4 inhibitor; viii) a SSTR5 receptor modulator, a GPR40 receptor modulator, a CCK_{A} receptor modulator, and a DPP-4 inhibitor; ix) a SSTR5 receptor modulator, a GPR119 receptor modulator, a CCK_{A} receptor modulator, and a DPP-4 inhibitor; or x) a GPR40 receptor modulator, a GPR119 receptor modulator, a CCK_{A} receptor modulator, and a DPP-4 inhibitor. In some embodiments, the method comprises administering to the individual: i) a TGR5 receptor modulator, a GPR40 receptor modulator, a GPR119 receptor modulator, a SSTR5 receptor modulator, and a DPP-4 inhibitor; ii) a TGR5 receptor modulator, a GPR40 receptor modulator, a GPR119 receptor modulator, a CCK_{A} receptor modulator, and a DPP-4 inhibitor; iii) a TGR5 receptor modulator, a GPR40 receptor modulator, a SSTR5 receptor modulator, a CCK_{A} receptor modulator, and a DPP-4 inhibitor; iv) a TGR5 receptor modulator, a GPR119 receptor modulator, a SSTR5 receptor modulator, a CCK_{A} receptor modulator, and a DPP-4 inhibitor; v) a GPR40 receptor modulator, a GPR119 receptor modulator, a SSTR5 receptor modulator, a CCK_{A} receptor modulator, and a DPP-4 inhibitor; or vi) a TGR5 receptor modulator, a GPR40 receptor modulator, a GPR119 receptor modulator, a SSTR5 receptor modulator, a CCK_{A} receptor modulator, and a DPP-4 inhibitor. In some embodiments, the method further comprises administering to the individual a PDE4 inhibitor. In some embodiments, the method comprises administering to the individual: i) a TGR5 receptor modulator, a SSTR5 receptor modulator, a PDE4 inhibitor, and a DPP-4 inhibitor; ii) a TGR5 receptor modulator, a GPR40 receptor modulator, a PDE4 inhibitor, and a DPP-4 inhibitor; iii) a TGR5 receptor modulator, a GPR119 receptor modulator, a PDE4 inhibitor, and a DPP-4 inhibitor; iv) a TGR5 receptor modulator, a CCK_{A} receptor modulator, a PDE4 inhibitor, and a DPP-4 inhibitor; v) a GPR40 receptor modulator, a GPR119 receptor modulator, a PDE4 inhibitor, and a DPP-4 inhibitor; vi) a GPR40 receptor modulator, a SSTR5 receptor modulator, a PDE4 inhibitor, and a DPP-4 inhibitor; vii) a GPR40 receptor modulator, a CCK_{A} receptor modulator, a PDE4 inhibitor, and a DPP-4 inhibitor; viii) a GPR119 receptor modulator, a SSTR5 receptor modulator, a PDE4 inhibitor, and a DPP-4 inhibitor; ix) a GPR119 receptor modulator, a CCK_{A} receptor modulator, a PDE4 inhibitor, and a DPP-4 inhibitor; or x) a SSTR5 receptor modulator, a CCK_{A} receptor modulator, a PDE4 inhibitor, and a DPP-4 inhibitor. In some embodiments, the method comprises administering to the individual: i) a TGR5 receptor modulator, a SSTR5 receptor modulator, a GPR119 receptor modulator, a PDE4 inhibitor, and a DPP-4 inhibitor; ii) a TGR5 receptor modulator, a SSTR5 receptor modulator, a GPR40 receptor modulator, a PDE4 inhibitor, and a DPP-4 inhibitor; iii) a TGR5 receptor modulator, a SSTR5 receptor modulator, a CCK_{A} receptor modulator, a PDE4 inhibitor, and a DPP-4 inhibitor; iv) a TGR5 receptor modulator, a GPR40 receptor modulator, a GPR119 receptor modulator, a PDE4 inhibitor, and a DPP-4 inhibitor; v) a TGR5 receptor modulator, a GPR40 receptor modulator, a CCK_{A} receptor modulator, a PDE4 inhibitor, and a DPP-4 inhibitor; vi) a TGR5 receptor modulator, a GPR119 receptor modulator, a CCK_{A} receptor modulator, a PDE4 inhibitor, and a DPP-4 inhibitor; vii) a GPR40 receptor modulator, a GPR119 receptor modulator, a SSTR5 receptor modulator, a PDE4 inhibitor, and a DPP-4 inhibitor; viii) a SSTR5 receptor modulator, a GPR40 receptor modulator, a CCK_{A} receptor modulator, a PDE4 inhibitor, and a DPP-4 inhibitor; ix) a SSTR5 receptor modulator, a GPR119 receptor modulator, a CCK_{A} receptor modulator, a PDE4 inhibitor, and a DPP-4 inhibitor; or x) a GPR40 receptor modulator, a GPR119 receptor modulator, a CCK_{A} receptor modulator, a PDE4 inhibitor, and a DPP-4 inhibitor. In some embodiments, the method comprises administering to the individual: i) a TGR5 receptor modulator, a GPR40 receptor modulator, a GPR119 receptor modulator, a SSTR5 receptor modulator, a PDE4 inhibitor, and a DPP-4 inhibitor; ii) a TGR5 receptor modulator, a GPR40 receptor modulator, a GPR119 receptor modulator, a CCK_{A} receptor modulator, a PDE4 inhibitor, and a DPP-4 inhibitor; iii) a TGR5 receptor modulator, a GPR40 receptor modulator, a SSTR5 receptor modulator, a CCK_{A} receptor modulator, a PDE4 inhibitor, and a DPP-4 inhibitor; iv) a TGR5 receptor modulator, a GPR119 receptor modulator, a SSTR5 receptor modulator, a CCK_{A} receptor modulator, a PDE4 inhibitor, and a DPP-4 inhibitor; v) a GPR40 receptor modulator, a GPR119 receptor modulator, a SSTR5 receptor modulator, a CCK_{A} receptor modulator, a PDE4 inhibitor, and a DPP-4 inhibitor; or vi) a TGR5 receptor modulator, a GPR40 receptor modulator, a GPR119 receptor modulator, a SSTR5 receptor modulator, a CCK_{A} receptor modulator, a PDE4 inhibitor, and a DPP-4 inhibitor.

In some embodiments, the TGR5 receptor modulator, the GPR40 receptor modulator, and GPR119 receptor modulator are gut-restricted modulators. In some embodiments, the TGR5 receptor modulator is a gut-restricted agonist of a TGR receptor. In some embodiments, the GPR40 receptor modulator is a gut-restricted agonist of a GPR40 receptor. In some embodiments, the GPR119 receptor modulator is a gut-restricted agonist of a GPR119 receptor.

In some embodiments, the SSTR5 receptor modulator is a gut-restricted antagonist of a SSTR5 receptor. In some embodiments, the SSTR5 receptor modulator is a gut-restricted inverse agonist of a SSTR5 receptor.

In some embodiments, the CCK_{A} receptor modulator is a gut-restricted agonist of a CCK_{A} receptor.

In some embodiments, the gut-restricted agonist, gut-restricted antagonist, or gut-restricted inverse agonist comprises a covalently bonded kinetophore, optionally through a linker. In some embodiments, at least two receptor modulators selected from the group consisting of a gut-restricted agonist, a gut-restricted antagonist, and a gut-restricted inverse agonist are covalently bonded, optionally through a linker. In some embodiments, the gut-restricted agonist, gut-restricted antagonist, or gut-restricted inverse agonist has <10% oral bioavailability, <8% oral bioavailability, <5% oral bioavailability, <3% oral bioavailability, or <2% oral bioavailability. In some embodiments, the unbound plasma levels of the gut-restricted agonist, gut-restricted antagonist, or gut-restricted inverse agonist is less than the IC₅₀ value or the EC₅₀ value of the gut-restricted agonist, gut-restricted antagonist, or gut-restricted inverse agonist against its receptor. In some embodiments, the unbound plasma levels of the gut-restricted agonist, gut-restricted antagonist, or gut-restricted inverse agonist are 2-fold, 10-fold, 20-fold, 50-fold, 100-fold lower than the IC₅₀ value or the EC₅₀ value of the gut-restricted agonist, gut-restricted antagonist, or gut-restricted inverse agonist against its receptor.

In some embodiments, the condition involving the gut-brain axis is a metabolic disorder. In some embodiments, the metabolic disorder is diabetes. In some embodiments, the metabolic disorder is obesity. In some embodiments, the condition involving the gut-brain axis is a nutritional disorder. In some embodiments, the nutritional disorder is short bowel syndrome, intestinal failure, or intestinal insufficiency. In some embodiments, the nutritional disorder is short bowel syndrome.

In some embodiments, disclosed herein is a pharmaceutical composition comprising at least two receptor modulators selected from the group consisting of a TGR5 receptor modulator, a GPR40 receptor modulator, a GPR119 receptor modulator, a somatostatin receptor 5 (SSTR5) modulator, and a CCK_{A} receptor modulator. In some embodiments, the pharmaceutical composition comprises: i) a TGR5 receptor modulator and a SSTR5 receptor modulator; ii) a TGR5 receptor modulator and a GPR40 receptor modulator; iii) a TGR5 receptor modulator and a GPR119 receptor modulator; iv) a TGR5 receptor modulator and a CCK_{A} receptor modulator; v) a GPR40 receptor modulator and a GPR119 receptor modulator; vi) a GPR40 receptor modulator and a SSTR5 receptor modulator; vii) a GPR40 receptor modulator and a CCK_{A} receptor modulator; viii) a GPR119 receptor modulator and a SSTR5 receptor modulator; ix) a GPR119 receptor modulator and a CCK_{A} receptor modulator; x) a SSTR5 receptor modulator and a CCK_{A} receptor modulator; xi) a TGR5 receptor modulator, a SSTR5 receptor modulator, and a GPR119 receptor modulator; xii) a TGR5 receptor modulator, a SSTR5 receptor modulator, and a GPR40 receptor modulator; xiii) a TGR5 receptor modulator, a SSTR5 receptor modulator, and a CCK_{A} receptor modulator; xiv) a TGR5 receptor modulator, a GPR40 receptor modulator, and a GPR119 receptor modulator; xv) a TGR5 receptor modulator, a GPR40 receptor modulator, and a CCK_{A} receptor modulator; xvi) a TGR5 receptor modulator, a GPR119 receptor modulator, and a CCK_{A} receptor modulator; xvii) a GPR40 receptor modulator, a GPR119 receptor modulator, and a SSTR5 receptor modulator; xviii) a SSTR5 receptor modulator, a GPR40 receptor modulator, and a CCK_{A} receptor modulator; xix) a SSTR5 receptor modulator, a GPR119 receptor modulator, and a CCK_{A} receptor modulator; xx) a GPR40 receptor modulator, a GPR119 receptor modulator, and a CCK_{A} receptor modulator; xxi) a TGR5 receptor modulator, a GPR40 receptor modulator, a GPR119 receptor modulator, and a SSTR5 receptor modulator; xxii) a TGR5 receptor modulator, a GPR40 receptor modulator, a GPR119 receptor modulator, and a CCK_{A} receptor modulator; xxiii) a TGR5 receptor modulator, a GPR40 receptor modulator, a SSTR5 receptor modulator, and a CCK_{A} receptor modulator; xxiv) a TGR5 receptor modulator, a GPR119 receptor modulator, a SSTR5 receptor modulator, and a CCK_{A} receptor modulator; xxv) a GPR40 receptor modulator, a GPR119 receptor modulator, and a SSTR5 receptor modulator, and a CCK_{A} receptor modulator; or xxvi) a TGR5 receptor modulator, a GPR40 receptor modulator, a GPR119 receptor modulator, a SSTR5 receptor modulator, and a CCK_{A} receptor modulator. In some embodiments the at least two receptor modulators are covalently bonded, optionally through a linker.

### BRIEF DESCRIPTION OF THE DRAWINGS

The features of the invention are set forth with particularity in the appended claims. A better understanding of the features of the present invention will be obtained by reference to the following detailed description that sets forth illustrative embodiments, in which the principles of the invention are utilized, and the accompanying drawings of which:

FIG. 1 depicts the effect of a GPR40 agonist, a GPR119 agonist, a TGR5 agonist, and combinations thereof on the body weight over a 10-day period in mice with diet induced obesity;

FIG. 2 depicts the effect of a GPR40 agonist, a GPR119 agonist, a TGR5 agonist, and combinations thereof on the total food consumption over a 9-day period in mice with diet induced obesity.

### DETAILED DESCRIPTION OF THE INVENTION

This disclosure is directed, at least in part, to GPCR modulators in combination therapies useful for the treatment of conditions or disorders involving the gut-brain axis. In some embodiments, these modulators are gut-restricted compounds. In some embodiments, these modulators are GPCR agonists, antagonists, inverse agonists, neutral antagonists, positive allosteric modulators, or negative allosteric modulators.

### Definitions

The term “modulate” or “modulating” or “modulation” refers to an increase or decrease in the amount, quality, or effect of a particular activity, function or molecule. By way of illustration and not limitation, agonists, inverse agonists, antagonists, and allosteric modulators of a G protein-coupled receptor are modulators of the receptor.

The term “agonism” as used herein refers to the activation of a receptor or enzyme by a modulator, or agonist, to produce a biological response.

The term “agonist” as used herein refers to a modulator that binds to a receptor or enzyme and activates the receptor to produce a biological response. By way of example only, “TGR5 agonist” can be used to refer to a compound that exhibits an EC₅₀ with respect to TGR5 activity of no more than about 100 μM, as measured in the cAMP production assay and glucagon-like peptide-1 (GLP-1) secretion assays. In some embodiments, the term “agonist” includes full agonists or partial agonists.

The term “full agonist” refers to a modulator that binds to and activates a receptor with the maximum response that an agonist can elicit at the receptor.

The term “partial agonist” refers to a modulator that binds to and activates a given receptor, but has partial efficacy, that is, less than the maximal response, at the receptor relative to a full agonist.

The term “positive allosteric modulator” refers to a modulator that binds to a site distinct from the orthosteric binding site and enhances or amplifies the effect of an agonist.

The term “antagonism” as used herein refers to the inactivation of a receptor or enzyme by a modulator, or antagonist. Antagonism of a receptor, for example, is when a molecule binds to the receptor and does not allow activity to occur.

The term “antagonist” or “neutral antagonist” as used herein refers to a modulator that binds to a receptor or enzyme and blocks a biological response. An antagonist has no activity in the absence of an agonist or inverse agonist but can block the activity of either, causing no change in the biological response.

The term “inverse agonist” refers to a modulator that binds to the same receptor as an agonist but induces a pharmacological response opposite to that agonist, i.e., a decrease in biological response.

The term “negative allosteric modulator” refers to a modulator that binds to a site distinct from the orthosteric binding site and reduces or dampens the effect of an agonist.

As used herein, “EC₅₀” is intended to refer to the concentration of a substance (e.g., a compound or a drug) that is required for 50% activation or enhancement of a biological process. In some instances, EC₅₀ refers to the concentration of agonist that provokes a response halfway between the baseline and maximum response in an in vitro assay. In some embodiments as used herein, EC₅₀ refers to the concentration of a modulator (e.g., an agonist) that is required for 50% activation of a GPCR, for example, TGR5, GPR40, or GPR119.

As used herein, “IC₅₀” is intended to refer to the concentration of a substance (e.g., a compound or a drug) that is required for 50% inhibition of a biological process. For example, IC₅₀ refers to the half maximal (50%) inhibitory concentration (IC) of a substance as determined in a suitable assay. In some instances, an IC₅₀ is determined in an in vitro assay system. In some embodiments as used herein, 1050 refers to the concentration of a modulator (e.g., an antagonist or inhibitor) that is required for 50% inhibition of a receptor, for example, SSTR5, TGR5, GPR40, or GPR119, or an enzyme, for example, DPP-4, or PDE4.

The terms “subject,” “individual,” and “patient” are used interchangeably. These terms encompass mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “gut-restricted” as used herein refers to a compound, e.g., a receptor modulator, that is predominantly active in the gastrointestinal system. In some embodiments, the biological activity of the gut-restricted compound, e.g., a gut-restricted receptor modulator, is restricted to the gastrointestinal system. In some embodiments, gastrointestinal concentration of a gut-restricted modulator, is higher than the IC₅₀ value or the EC₅₀ value of the gut-restricted modulator against its receptor, while the plasma levels of said gut-restricted modulator are lower than the IC₅₀ value or the EC₅₀ value of the gut-restricted modulator against its receptor. In some embodiments, the gut-restricted compound, e.g., a receptor modulator, is non-systemic. In some embodiments, the gut-restricted compound, e.g., a receptor modulator, is a non-absorbed compound. In other embodiments, the gut-restricted compound is absorbed, but is rapidly metabolized to metabolites that are significantly less active than the modulator itself toward the target receptor, i.e., a “soft drug.” In other embodiments, the gut-restricted compound is minimally absorbed and rapidly metabolized to metabolites that are significantly less active than the modulator itself toward the target receptor.

In some embodiments, the gut-restricted modulator is non-systemic but is instead localized to the gastrointestinal system. In some instances, the modulator is present in high levels in the gut, but low levels in serum. In some embodiments, the systemic exposure of a gut-restricted modulator is, for example, less than 100, less than 50, less than 20, less than 10, or less than 5 nM, bound or unbound, in blood serum. In some embodiments, the intestinal exposure of a gut-restricted modulator is, for example, greater than 1000, 5000, 10000, 50000, 100000, or 500000 nM. In some embodiments, a modulator is gut-restricted due to poor absorption of the modulator itself, or because of absorption of the modulator which is rapidly metabolized in serum resulting in low systemic circulation, or due to both poor absorption and rapid metabolism in the serum. In some embodiments, a modulator is covalently bonded to a kinetophore, optionally through a linker, which changes the pharmacokinetic profile of the modulator. In other embodiments, two or more modulators are covalently bonded, optionally through a linker, to each other.

In other embodiments, the gut-restricted modulator is a soft drug. The term “soft drug” as used herein refers to a modulator that is biologically active but is rapidly metabolized to metabolites that are significantly less active than the modulator itself toward the target receptor. In some embodiments, the gut-restricted modulator is a soft drug that is rapidly metabolized in the blood to significantly less active metabolites. In some embodiments, the gut-restricted modulator is a soft drug that is rapidly metabolized in the liver to significantly less active metabolites. In some embodiments, the gut-restricted modulator is a soft drug that is rapidly metabolized in the blood and the liver to significantly less active metabolites. In some embodiments, the gut-restricted modulator is a soft drug that has low systemic exposure. In some embodiments, the biological activity of the metabolite(s) is/are 10-fold, 20-fold, 50-fold, 100-fold, 500-fold, or 1000-fold lower than the biological activity of the soft drug gut-restricted modulator.

The term “kinetophore” as used herein refers to a structural unit tethered to a small molecule modulator, optionally through a linker, which makes the whole molecule larger and increases the polar surface area while maintaining biological activity of the small molecule modulator. The kinetophore influences the pharmacokinetic properties, for example solubility, absorption, distribution, rate of elimination, and the like, of the small molecule modulator and has minimal changes to the binding to or association with a receptor. The defining feature of a kinetophore is not its interaction with the target, for example a receptor, but rather its effect on specific physiochemical characteristics of the modulator to which it is attached. In some instances, kinetophores are used to restrict a modulator to the gut.

The term “linked” as used herein refers to a covalent linkage between a modulator and a kinetophore or between a modulator and at least one other modulator, or a combination thereof. The linkage can be through a covalent bond, or through a “linker.” As used herein, “linker” refers to one or more bifunctional molecules which can be used to covalently bond to the modulator(s) and/or kinetophore. In some embodiments, the linker is attached to any part of the modulator so long as the point of attachment does not interfere with the binding of the modulator to its receptor. In some embodiments, the linker is non-cleavable. In some embodiments, the linker is cleavable. In some embodiments, the linker is cleavable in the gut. In some embodiments, cleaving the linker releases the biologically active modulator in the gut.

The term “gastrointestinal system” (GI system) or “gastrointestinal tract” (GI tract) as used herein, refers to the organs and systems involved in the process of digestion. The gastrointestinal tract includes the esophagus, stomach, small intestine, which includes the duodenum, jejunum, and ileum, and large intestine, which includes the cecum, colon, and rectum. In some embodiments herein, the GI system refers to the “gut,” meaning the stomach, small intestines, and large intestines or to the small and large intestines, including, for example, the duodenum, jejunum, and/or colon.

### Gut-Brain Axis

The gut-brain axis refers to the bidirectional biochemical signaling that connects the gastrointestinal tract (GI tract) with the central nervous system (CNS) through the peripheral nervous system (PNS) and endocrine, immune, and metabolic pathways.

In some instances, the gut-brain axis comprises the GI tract; the PNS including the dorsal root ganglia (DRG) and the sympathetic and parasympathetic arms of the autonomic nervous system including the enteric nervous system and the vagus nerve; the CNS; and the neuroendocrine and neuroimmune systems including the hypothalamic-pituitary-adrenal axis (HPA axis). The gut-brain axis is important for maintaining homeostasis of the body and is regulated and modulates physiology through the central and peripheral nervous systems and endocrine, immune, and metabolic pathways.

The gut-brain axis modulates several important aspects of physiology and behavior. Modulation by the gut-brain axis occurs via hormonal and neural circuits. Key components of these hormonal and neural circuits of the gut-brain axis include highly specialized, secretory intestinal cells that release hormones (enteroendocrine cells or EECs), the autonomic nervous system (including the vagus nerve and enteric nervous system), and the central nervous system. These systems work together in a highly coordinated fashion to modulate physiology and behavior.

Defects in the gut-brain axis are linked to a number of diseases, including those of high unmet need. Diseases and conditions affected by the gut-brain axis, include central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, celiac disease, and enteritis, including chemotherapy-induced enteritis or radiation-induced enteritis; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis.

### G-Protein Coupled Receptors (GPCRs) in the Gut-Brain Axis

GPCRs play many important roles in the gut-brain axis for sensing and relaying food-related signals, physiologic signals such as stretch and pH, and signals arising from the microbiome and microbiome metabolites. In some instances, GPCRs are key to the coordination of appetite, digestion, and nutrient disposal, and contribute at a number of levels to the recruitment and integration of pathways linking the gut, brain, and peripheral tissues. In some instances, GPCRs in enteroendocrine cells act as activators or inhibitors of endogenous release of GLP-1, GLP-2, GIP, PYY, CCK, and other hormones. In some instances, these GPCRs include TGR5, GPR40, GPR119, and SSTR5. In some instances, GPCRs control appetite through activation of the vagus nerve. In some instances these GPCRs include CCK_{A}. In some embodiments described herein, modulation of a combination of GPCRs in the gut is useful for the treatment of conditions or disorders involving the gut-brain axis.

### TGR5

The G protein-coupled bile acid receptor 1 (GPBAR1), also known G-protein coupled receptor 19 (GPCR19), membrane-type receptor for bile acids (M-BAR) or Takeda G-protein-coupled receptor 5 (TGR5), is a member of the GPCR superfamily. This protein functions as a cell surface receptor for bile acids. In some instances, TGR5 is expressed in the gall bladder, brown adipose tissue, muscle, liver, the central nervous system, and intestinal enteroendocrine cells. In some instances, TGR5 is expressed in intestinal enteroendocrine cells. Upon binding bile acid, the production of intracellular cAMP and activation of a MAP kinase signaling pathway is induced in cells expressing TGR5. In some instances, TGR-5 agonists are useful in the treatment of metabolic diseases (such as obesity, diabetes and NASH), inflammatory diseases (such as IBD), gut diseases (such as short bowel syndrome), and other diseases involving the gut-brain axis.

In some instances, modulators of TGR5, for example, TGR5 agonists, induce the production of intracellular cAMP. In some instances, modulators of TGR5, for example, TGR5 agonists, induce the secretion of glucagon-like peptide-1 (GLP-1), glucagon-like peptide-2 (GLP-2), glucose-dependent insulinotropic polypeptide (GIP), peptide YY (PYY), cholecystokinin (CCK), or other hormones. In some instances, modulators of TGR5, for example, TGR5 agonists, induce the secretion of GLP-1, GIP, CCK or PYY. In some instances, modulators of TGR5, for example, TGR5 agonists, induce the secretion of GLP-1.

Described herein is a method of treating a condition or disorder involving the gut-brain axis in an individual in need thereof, the method comprising administering to the individual a TGR5 receptor modulator. In some embodiments, the TGR5 receptor modulator is a TGR5 agonist. In some embodiments, the TGR5 receptor modulator is a TGR5 agonist. In some embodiments, the TGR5 receptor modulator is a TGR5 positive allosteric modulator. In some embodiments, the TGR5 modulator is administered in combination with a GPR40 receptor modulator, a GPR119 receptor modulator, or a somatostatin receptor 5 (SSTR5) modulator, or a combination thereof. In some embodiments, the TGR5 receptor modulator is administered with a PDE4 inhibitor, a DPP-4 inhibitor, or a combination thereof.

### GPR40

Free fatty acid receptor 1 (FFA1, FFAR1), also known as GPR40, is a class A G-protein coupled receptor. This membrane protein binds free fatty acids, acting as a nutrient sensor for regulating energy homeostasis. In some instances, GPR40 is expressed in enteroendocrine cells and pancreatic islet β cells. In some instances, GPR40 is expressed in enteroendocrine cells. Several naturally-occurring medium to long-chain fatty acids act as ligands for GPR40. In some instances, GPR40 agonists are useful in the treatment of metabolic diseases (such as obesity, diabetes, and NASH) and other diseases involving the gut-brain axis.

In some instances, modulators of GPR40, for example, GPR40 agonists, induce insulin secretion. In some instances, modulators of GPR40, for example, GPR40 agonists, induce an increase in cytosolic Ca′. In some instances, modulators of GPR40, for example, GPR40 agonists, induce higher levels of intracellular cAMP. In some instances, GPR40 modulation is in enteroendocrine cells. In some instances, modulators of GPR40, for example, GPR40 agonists, induce the secretion of GLP-1, GLP-2, GIP, PYY, CCK, or other hormones. In some instances, modulators of GPR40, for example, GPR40 agonists, induce the secretion of GLP-1, GIP, CCK or PYY. In some instances, modulators of GPR40, for example, GPR40 agonists, induce the secretion of GLP-1.

Described herein is a method of treating a condition or disorder involving the gut-brain axis in an individual in need thereof, the method comprising administering to the individual a GPR40 receptor modulator. In some embodiments, the GPR40 receptor modulator is a GPR40 full agonist. In some embodiments, the GPR40 receptor modulator is a GPR40 partial agonist. In some embodiments, the GPR40 receptor modulator is a GPR40 positive allosteric modulator. In some embodiments, the GPR40 modulator is administered in combination with a TGR5 receptor modulator, a GPR119 receptor modulator, or a somatostatin receptor 5 (SSTR5) modulator, or a combination thereof. In some embodiments, the GPR40 receptor modulator is administered with a PDE4 inhibitor, a DPP-4 inhibitor, or a combination thereof.

### GPR119

GPR119 is a Class A G protein-coupled receptor. In some instances, GPR119 is expressed in the pancreas and in enteroendocrine cells of the gastrointestinal tract. In some instances, GPR119 is expressed in enteroendocrine cells. GPR119 is activated by oleoylethanolamide (OEA) and other oleic acid derivatives and N-acylethanolamides. In some instances, GPR119 agonists are useful in the treatment of metabolic diseases (such as diabetes and obesity), and other diseases involving the gut-brain axis.

In some instances, modulators of GPR119, for example, GPR119 agonists, induce the production of intracellular cAMP. In some instances, modulators of GPR119, for example, GPR119 agonists, induce the secretion of GLP-1, GLP-2, GIP, PYY, CCK, or other hormones. In some instances, modulators of GPR119, for example, GPR119 agonists, induce the secretion of GLP-1, GIP, CCK or PYY. In some instances, modulators of GPR119, for example, GPR119 agonists, induce the secretion of GLP-1.

Described herein is a method of treating a condition or disorder involving the gut-brain axis in an individual in need thereof, the method comprising administering to the individual a GPR119 receptor modulator. In some embodiments, the GPR119 receptor modulator is a GPR119 agonist or partial agonist. In some embodiments, the GPR119 receptor modulator is a GPR119 agonist. In some embodiments, the GPR119 receptor modulator is a GPR119 positive allosteric modulator. In some embodiments, the GPR119 modulator is administered in combination with a TGR5 receptor modulator, a GPR40 receptor modulator, or a somatostatin receptor 5 (SSTR5) modulator, or a combination thereof. In some embodiments, the GPR119 receptor modulator is administered with a PDE4 inhibitor, a DPP-4 inhibitor, or a combination thereof.

### SSTR5

Somatostatin acts at many sites to inhibit the release of many hormones and other secretory proteins. Somatostatin is predominantly expressed in two forms, SST-14 in foregut and enteric nervous system and SST-28 in the ileum, colon and pancreatic [symbol] -cells. In some instances, the biological effects of somatostatin are mediated by a family of G protein-coupled receptors that are expressed in a tissue-specific manner. SSTR5 is a member of the superfamily of receptors and is expressed on β cells of pancreatic islets, GI epithelium and enteroendocrine cells, and cardiac tissue. In some instances, somatostatin binding to SSTR5 inhibits the release of GLP-1, GLP-2, GIP, PYY, CCK, or other hormones in enteroendocrine cells. In some instances, SSTR5 antagonists are useful in the treatment of metabolic disorders (such as diabetes and obesity), and other diseases involving the gut-brain axis.

In some instances, inhibiting SSTR5 activity results in an elevated level of GLP-1, GLP-2, GIP, PYY, CCK, and other hormones in enteroendocrine cells. In some instances, modulators of SSTR5, for example, SSTR5 antagonists, facilitate the release of GLP-1, GLP-2, GIP, PYY, CCK, and other hormones in enteroendocrine cells by blocking the activity of somatostatin. In some instances, modulators of SSTR5, for example, SSTR5 antagonists, lead to increased cAMP levels by blocking the activity of somatostatin. In some instances, SSTR5 activity, upon binding of somatostatin, inhibits intracellular cAMP production and GLP-1, GLP-2, GIP, PYY, CCK and other hormone secretion. In some instances, inhibiting SSTR5 activity results in elevated intracellular cAMP levels and elevated GLP-1, GIP, PYY, CCK or other hormone secretion. In some instances, inhibiting SSTR5 activity results in elevated intracellular cAMP levels and elevated GLP-1 secretion.

Described herein is a method of treating a condition or disorder involving the gut-brain axis in an individual in need thereof, the method comprising administering to the individual a SSTR5 receptor modulator. In some embodiments, the SSTR5 receptor modulator is a SSTR5 antagonist or SSTR5 inverse agonist. In some embodiments, the SSTR5 receptor modulator is a SSTR5 antagonist. In some embodiments, the SSTR5 receptor modulator is a SSTR5 inverse agonist. In some embodiments, the SSTR5 receptor modulator is a SSTR5 negative allosteric modulator. In some embodiments, the SSTR5 modulator is administered in combination with a TGR receptor modulator, a GPR40 receptor modulator, or a GPR119 modulator, or a combination thereof. In some embodiments, the SSTR5 receptor modulator is administered with a PDE4 inhibitor, a DPP-4 inhibitor, or a combination thereof.

### CCK_{A} Receptors

Cholecystokinin receptors, or CCK receptors, are a group of G-protein coupled receptors which bind the peptide hormones cholecystokinin (CCK) and gastrin. The CCK receptors are found in the gastrointestinal tract and in the CNS. In some instances, cholecystokinin A receptor (CCKAR, CCK1, CCK_{A}) is found primarily in the gastrointestinal tract. In some instances, CCKAR is activated by sulfated members of the CCK family of peptide hormones. In some instances, CCK_{A} agonists are useful in the treatment of metabolic diseases (such as diabetes and obesity), and other diseases involving the gut-brain axis.

### Peptide Hormones

Incretins are a group of metabolic hormones released in the gut that stimulate a decrease in blood glucose levels in a glucose-dependent manner. Incretins include the peptide hormones GLP-1 and GIP. In some instances, incretin hormones are released in enteroendocrine cells after eating. In some instances, incretin hormones augment the secretion of insulin released from pancreatic beta cells of the islets of Langerhans by a blood glucose-dependent mechanism. In some instances, incretin hormones (such as GLP-1) also inhibit glucagon release from the alpha cells of the islets of Langerhans. Beside insulinotropic effects, GLP-1 has been associated with numerous regulatory and protective effects. GLP-1 inhibits gastric emptying, acid secretion, motility, decreases appetite and promotes satiety. GLP-1 receptor activation has been linked with neurotrophic effects including neurogenesis and neuroprotective effects including reduced necrotic and apoptotic signalling and cell death. GLP-1 receptor agonist treatment is associated with protection against a range of experimental disease models such as Parkinson's disease, Alzheimer's disease, stroke, traumatic brain injury, and multiple sclerosis. Other peptide hormones released in the gut include CCK, PYY, GLP-2, oxyntomdulin, gastrin, secretin, vasoactive intestinal peptide (VIP), motilin, ghrelin, bombesin, calcitonin gene-related peptide (CGRP), chromogranin A, enkephalins, enteroglucagon, galanin, ghrelin, growth factors, growth hormone-releasing factor, leptin, motilin, amylin, neuropeptide Y (NPY), neurotensin, pancreatic polypeptide, somatostatin, substance P and trefoil peptides. These peptides regulate a wide variety of processes including food intake, metabolic rate, glucose homeostasis, gastric emptying, gut motility, gall bladder contraction, pancreatic secretion, intestinal mucosal growth, muscosal protection and repair, pain, cell proliferation and differentiation, water and electrolyte secretion, and intestinal blood flow.

In some instances, modulating the activity of the GPCRs described herein, e.g., TGR5, GPR40, GPR119, and SSTR5 increases peptide hormone secretion. In some instances, the biological effect of peptide hormones is in enteroendocrine cells. In some instances, peptide hormones, (e.g., GLP-1 and GIP), stimulate insulin release in a glucose dependent manner. In some instances, GLP-1, for example, is necessary for normal glucose homeostasis. In some instances, peptide hormones, (e.g., GLP-1, GLP-2 and GIP), contribute to beneficial effects for the treatment of diseases or conditions involving the gut-brain axis (e.g., diabetes, obesity or short bowel syndrome), including 1) increased insulin secretion, 2) increased glucose disposal, 3) suppression in glucose production, 4) reduced gastric emptying, 5) reduction in food intake, 6) body mass reduction, 7) increased cAMP levels, 8) increased nutrient absorption, 9) increased small intestinal length, 10) increased small intestinal weight, 11) increased villus height, and 12) increased villus height/crypt depth ratio.

### Combination Therapies

In some embodiments, disclosed herein is method of treating a condition or disorder involving the gut-brain axis in an individual in need thereof, the method comprising administering to the individual at least two receptor modulators selected from the group consisting of a TGR5 receptor modulator, a GPR40 receptor modulator, a GPR119 receptor modulator, and a somatostatin receptor 5 (SSTR5) modulator. In some embodiments, the method comprises administering to the individual at least three receptor modulators selected from the group consisting of a TGR5 receptor modulator, a GPR40 receptor modulator, a GPR119 receptor modulator, and a somatostatin receptor 5 (SSTR5) modulator. In some embodiments, the method comprises administering to the individual at least four receptor modulators selected from the group consisting of a TGR5 receptor modulator, a GPR40 receptor modulator, a GPR119 receptor modulator, and a somatostatin receptor 5 (SSTR5) modulator. In some embodiments, at least one of the group consisting of i) the TGR5 receptor modulator, ii) the GPR40 receptor modulator, and iii) the GPR119 receptor modulator is a gut-restricted modulator. In some embodiments, at least one of the group consisting of i) the TGR5 receptor modulator, ii) the GPR40 receptor modulator, iii) the GPR119 receptor modulator, and iv) the somatostatin receptor 5 (SSTR5) modulator is a gut-restricted modulator.

In some embodiments, disclosed herein is method of treating a condition or disorder involving the gut-brain axis in an individual in need thereof, the method comprising administering to the individual at least two receptor modulators selected from the group consisting of a TGR5 receptor modulator, a GPR40 receptor modulator, a GPR119 receptor modulator, and a somatostatin receptor 5 (SSTR5) modulator; wherein at least one of the group consisting of i) the TGR5 receptor modulator, ii) the GPR40 receptor modulator, and iii) the GPR119 receptor modulator is a gut-restricted modulator. In some embodiments, at least one of the group consisting of i) the TGR5 receptor modulator, ii) the GPR40 receptor modulator, iii) the GPR119 receptor modulator, and iv) the somatostatin receptor 5 (SSTR5) modulator is a gut-restricted modulator.

In some embodiments, disclosed herein is method of treating a condition or disorder involving the gut-brain axis in an individual in need thereof, the method comprising administering to the individual at least two receptor modulators selected from the group consisting of a TGR5 agonist, a GPR40 agonist, a GPR119 agonist, and a somatostatin receptor 5 (SSTR5) antagonist. In some embodiments, the method comprises administering to the individual at least three receptor modulators selected from the group consisting of a TGR5 agonist, a GPR40 agonist, a GPR119 agonist, and a somatostatin receptor 5 (SSTR5) antagonist. In some embodiments, the method comprises administering to the individual at least four receptor modulators selected from the group consisting of a TGR5 agonist, a GPR40 agonist, a GPR119 agonist, and a somatostatin receptor 5 (SSTR5) antagonist. In some embodiments, at least one of the group consisting of i) the TGR5 agonist, ii) the GPR40 agonist, and iii) the GPR119 agonist is a gut-restricted agonist. In some embodiments, at least one of the group consisting of i) the TGR5 agonist, ii) the GPR40 agonist, iii) the GPR119 agonist, and iv) the somatostatin receptor 5 (SSTR5) antagonist is a gut-restricted modulator.

In some embodiments, disclosed herein is method of treating a condition or disorder involving the gut-brain axis in an individual in need thereof, the method comprising administering to the individual at least two receptor modulators selected from the group consisting of a TGR5 agonist, a GPR40 agonist, a GPR119 agonist, and a somatostatin receptor 5 (SSTR5) antagonist; wherein at least one of the group consisting of i) the TGR5 agonist, ii) the GPR40 agonist, and iii) the GPR119 agonist is a gut-restricted agonist. In some embodiments, at least one of the group consisting of i) the TGR5 agonist, ii) the GPR40 agonist, iii) the GPR119 agonist, and iv) the somatostatin receptor 5 (SSTR5) antagonist is a gut-restricted modulator.

In some embodiments, the method comprises administering to the individual: i) a TGR5 receptor modulator and a SSTR5 receptor modulator; ii) a TGR5 receptor modulator and a GPR40 receptor modulator; iii) a TGR5 receptor modulator and a GPR119 receptor modulator; iv) a GPR40 receptor modulator and a GPR119 receptor modulator; v) a GPR40 receptor modulator and a SSTR5 receptor modulator; or vi) a GPR119 receptor modulator and a SSTR5 receptor modulator. In some embodiments, the method comprises administering to the individual a TGR5 receptor modulator and a SSTR5 receptor modulator. In some embodiments, the method comprises administering to the individual a TGR5 receptor modulator and a GPR40 receptor modulator. In some embodiments, the method comprises administering to the individual a TGR5 receptor modulator and a GPR119 receptor modulator. In some embodiments, the method comprises administering to the individual a GPR40 receptor modulator and a GPR119 receptor modulator. In some embodiments, the method comprises administering to the individual a GPR40 receptor modulator and a SSTR5 receptor modulator. In some embodiments, the method comprises administering to the individual a GPR119 receptor modulator and a SSTR5 receptor modulator.

In some embodiments, the method comprises administering to the individual: i) a TGR5 receptor modulator, a SSTR5 receptor modulator, and a GPR119 receptor modulator; ii) a TGR5 receptor modulator, a SSTR5 receptor modulator, and a GPR40 receptor modulator; iii) a TGR5 receptor modulator, a GPR40 receptor modulator, and a GPR119 receptor modulator; iv) a GPR40 receptor modulator, a GPR119 receptor modulator, and a SSTR5 receptor modulator; or v) a TGR5 receptor modulator, a GPR40 receptor modulator, a GPR119 receptor modulator, and a SSTR5 receptor modulator. In some embodiments, the method comprises administering to the individual a TGR5 receptor modulator, a SSTR5 receptor modulator, and a GPR119 receptor modulator. In some embodiments, the method comprises administering to the individual a TGR5 receptor modulator, a SSTR5 receptor modulator, and a GPR40 receptor modulator. In some embodiments, the method comprises administering to the individual a TGR5 receptor modulator, a GPR40 receptor modulator, and a GPR119 receptor modulator. In some embodiments, the method comprises administering to the individual a GPR40 receptor modulator, a GPR119 receptor modulator, and a SSTR5 receptor modulator. In some embodiments, the method comprises administering to the individual a TGR5 receptor modulator, a GPR40 receptor modulator, a GPR119 receptor modulator, and a SSTR5 receptor modulator.

In some embodiments, the method comprises administering to the individual: i) a TGR5 receptor modulator and a GPR40 receptor modulator; ii) a TGR5 receptor modulator and a GPR119 receptor modulator; or iii) a GPR40 receptor modulator and a GPR119 receptor modulator. In some embodiments, the method comprises administering to the individual TGR5 receptor modulator and a GPR40 receptor modulator. In some embodiments, the method comprises administering to the individual a TGR5 receptor modulator and a GPR119 receptor modulator. In some embodiments, the method comprises administering to the individual a GPR40 receptor modulator and a GPR119 receptor modulator.

In some embodiments, the method comprises administering to the individual a TGR5 receptor modulator, a GPR40 receptor modulator, and a GPR119 receptor modulator. In some embodiments, the method further comprises administering to the individual a SSTR5 receptor modulator. In some embodiments, the method comprises administering to the individual a TGR5 receptor modulator, a GPR40 receptor modulator, a GPR119 receptor modulator, and a SSTR5 receptor modulator.

In some embodiments, the method further comprises administering to the individual a phosphodiesterase type 4 (PDE4) inhibitor. In some instances, PDE4 hydrolyzes intracellular cAMP, leading to lower intracellular cAMP levels. In some instances, use of a PDE4 inhibitor potentiates cAMP levels associated GPCR agonism. In some instances, use of a PDE4 inhibitor maintains the elevated cAMP levels associated GPCR agonism. In some instances, use of a PDE4 inhibitor lengthens the time which cAMP levels are elevated through GPCR agonism.

In some embodiments, the method comprises administering to the individual: i) a TGR5 receptor modulator, a SSTR5 receptor modulator, and a PDE4 inhibitor; ii) a TGR5 receptor modulator, a GPR40 receptor modulator, and a PDE4 inhibitor; iii) a TGR5 receptor modulator, a GPR119 receptor modulator, and a PDE4 inhibitor; iv) a GPR40 receptor modulator, a GPR119 receptor modulator, and a PDE4 inhibitor; v) a GPR40 receptor modulator, a SSTR5 receptor modulator, and a PDE4 inhibitor; or vi) a GPR119 receptor modulator, a SSTR5 receptor modulator, and a PDE4 inhibitor. In some embodiments, the method comprises administering to the individual a TGR5 receptor modulator, a SSTR5 receptor modulator, and a PDE4 inhibitor. In some embodiments, the method comprises administering to the individual a TGR5 receptor modulator, a GPR40 receptor modulator, and a PDE4 inhibitor. In some embodiments, the method comprises administering to the individual a TGR5 receptor modulator, a GPR119 receptor modulator, and a PDE4 inhibitor. In some embodiments, the method comprises administering to the individual a GPR40 receptor modulator, a GPR119 receptor modulator, and a PDE4 inhibitor. In some embodiments, the method comprises administering to the individual a GPR40 receptor modulator, a SSTR5 receptor modulator, and a PDE4 inhibitor. In some embodiments, the method comprises administering to the individual a GPR119 receptor modulator, a SSTR5 receptor modulator, and a PDE4 inhibitor.

In some embodiments, the method comprises administering to the individual: i) a TGR5 receptor modulator, a SSTR5 receptor modulator, a GPR119 receptor modulator, and a PDE4 inhibitor; ii) a TGR5 receptor modulator, a SSTR5 receptor modulator, a GPR40 receptor modulator, and a PDE4 inhibitor; iii) a TGR5 receptor modulator, a GPR40 receptor modulator, a GPR119 receptor modulator, and a PDE4 inhibitor; iv) a GPR40 receptor modulator, a GPR119 receptor modulator, a SSTR5 receptor modulator, and a PDE4 inhibitor; or v) a TGR5 receptor modulator, a GPR40 receptor modulator, a GPR119 receptor modulator, a SSTR5 receptor modulator, and a PDE4 inhibitor. In some embodiments, the method comprises administering to the individual a TGR5 receptor modulator, a SSTR5 receptor modulator, a GPR119 receptor modulator, and a PDE4 inhibitor. In some embodiments, the method comprises administering to the individual a TGR5 receptor modulator, a SSTR5 receptor modulator, a GPR40 receptor modulator, and a PDE4 inhibitor. In some embodiments, the method comprises administering to the individual a TGR5 receptor modulator, a GPR40 receptor modulator, a GPR119 receptor modulator, and a PDE4 inhibitor. In some embodiments, the method comprises administering to the individual a GPR40 receptor modulator, a GPR119 receptor modulator, a SSTR5 receptor modulator, and a PDE4 inhibitor. In some embodiments, the method comprises administering to the individual a TGR5 receptor modulator, a GPR40 receptor modulator, a GPR119 receptor modulator, a SSTR5 receptor modulator, and a PDE4 inhibitor.

In some embodiments, the method further comprises administering to the individual a dipeptidyl peptidase-4 (DPP-4) inhibitor. In some instances, DPP-4 plays a role in the degradation of signaling hormones, for example GLP-1 and GIP. In some instances, use of a DPP-4 inhibitor potentiates active hormone levels associated GPCR agonism. In some instances, use of a DPP-4 inhibitor maintains the elevated hormone secretion levels associated GPCR agonism. In some instances, use of a DPP-4 inhibitor lengthens the time which hormone secretion is elevated through GPCR agonism.

In some embodiments, the method comprises administering to the individual: i) a TGR5 receptor modulator, a SSTR5 receptor modulator, and a DPP-4 inhibitor; ii) a TGR5 receptor modulator, a GPR40 receptor modulator, and a DPP-4 inhibitor; iii) a TGR5 receptor modulator, a GPR119 receptor modulator, and a DPP-4 inhibitor; iv) a GPR40 receptor modulator, a GPR119 receptor modulator, and a DPP-4 inhibitor; v) a GPR40 receptor modulator, a SSTR5 receptor modulator, and a DPP-4 inhibitor; or vi) a GPR119 receptor modulator, a SSTR5 receptor modulator, and a DPP-4 inhibitor. In some embodiments, the method comprises administering to the individual a TGR5 receptor modulator, a SSTR5 receptor modulator, and a DPP-4 inhibitor. In some embodiments, the method comprises administering to the individual a TGR5 receptor modulator, a GPR40 receptor modulator, and a DPP-4 inhibitor. In some embodiments, the method comprises administering to the individual a TGR5 receptor modulator, a GPR119 receptor modulator, and a DPP-4 inhibitor. In some embodiments, the method comprises administering to the individual a GPR40 receptor modulator, a GPR119 receptor modulator, and a DPP-4 inhibitor. In some embodiments, the method comprises administering to the individual a GPR40 receptor modulator, a SSTR5 receptor modulator, and a DPP-4 inhibitor. In some embodiments, the method comprises administering to the individual a GPR119 receptor modulator, a SSTR5 receptor modulator, and a DPP-4 inhibitor.

In some embodiments, the method comprises administering to the individual: i) a TGR5 receptor modulator, a SSTR5 receptor modulator, a GPR119 receptor modulator, and a DPP-4 inhibitor; ii) a TGR5 receptor modulator, a SSTR5 receptor modulator, a GPR40 receptor modulator, and a DPP-4 inhibitor; iii) a TGR5 receptor modulator, a GPR40 receptor modulator, a GPR119 receptor modulator, and a DPP-4 inhibitor; iv) a GPR40 receptor modulator, a GPR119 receptor modulator, a SSTR5 receptor modulator, and a DPP-4 inhibitor; or v) a TGR5 receptor modulator, a GPR40 receptor modulator, a GPR119 receptor modulator, a SSTR5 receptor modulator, and a DPP-4 inhibitor. In some embodiments, the method comprises administering to the individual a TGR5 receptor modulator, a SSTR5 receptor modulator, a GPR119 receptor modulator, and a DPP-4 inhibitor. In some embodiments, the method comprises administering to the individual a TGR5 receptor modulator, a SSTR5 receptor modulator, a GPR40 receptor modulator, and a DPP-4 inhibitor. In some embodiments, the method comprises administering to the individual a TGR5 receptor modulator, a GPR40 receptor modulator, a GPR119 receptor modulator, and a DPP-4 inhibitor. In some embodiments, the method comprises administering to the individual a GPR40 receptor modulator, a GPR119 receptor modulator, a SSTR5 receptor modulator, and a DPP-4 inhibitor. In some embodiments, the method comprises administering to the individual a TGR5 receptor modulator, a GPR40 receptor modulator, a GPR119 receptor modulator, a SSTR5 receptor modulator, and a DPP-4 inhibitor.

In some embodiments, the method comprises administering to the individual: i) a TGR5 receptor modulator, a SSTR5 receptor modulator, a PDE4 inhibitor, and a DPP-4 inhibitor; ii) a TGR5 receptor modulator, a GPR40 receptor modulator, a PDE4 inhibitor, and a DPP-4 inhibitor; iii) a TGR5 receptor modulator, a GPR119 receptor modulator, a PDE4 inhibitor, and a DPP-4 inhibitor; iv) a GPR40 receptor modulator, a GPR119 receptor modulator, a PDE4 inhibitor, and a DPP-4 inhibitor; v) a GPR40 receptor modulator, a SSTR5 receptor modulator, a PDE4 inhibitor, and a DPP-4 inhibitor; or vi) a GPR119 receptor modulator, a SSTR5 receptor modulator, a PDE4 inhibitor and a DPP-4 inhibitor. In some embodiments, the method comprises administering to the individual a TGR5 receptor modulator, a SSTR5 receptor modulator, a PDE4 inhibitor, and a DPP-4 inhibitor. In some embodiments, the method comprises administering to the individual a TGR5 receptor modulator, a GPR40 receptor modulator, a PDE4 inhibitor, and a DPP-4 inhibitor. In some embodiments, the method comprises administering to the individual a TGR5 receptor modulator, a GPR119 receptor modulator, a PDE4 inhibitor, and a DPP-4 inhibitor. In some embodiments, the method comprises administering to the individual a GPR40 receptor modulator, a GPR119 receptor modulator, a PDE4 inhibitor, and a DPP-4 inhibitor. In some embodiments, the method comprises administering to the individual a GPR40 receptor modulator, a SSTR5 receptor modulator, a PDE4 inhibitor, and a DPP-4 inhibitor. In some embodiments, the method comprises administering to the individual a GPR119 receptor modulator, a SSTR5 receptor modulator, a PDE4 inhibitor and a DPP-4 inhibitor.

In some embodiments, the method comprises administering to the individual: i) a TGR5 receptor modulator, a SSTR5 receptor modulator, a GPR119 receptor modulator, a PDE4 inhibitor, and a DPP-4 inhibitor; ii) a TGR5 receptor modulator, a SSTR5 receptor modulator, a GPR40 receptor modulator, a PDE4 inhibitor, and a DPP-4 inhibitor; iii) a TGR5 receptor modulator, a GPR40 receptor modulator, a GPR119 receptor modulator, a PDE4 inhibitor, and a DPP-4 inhibitor; iv) a GPR40 receptor modulator, a GPR119 receptor modulator, a SSTR5 receptor modulator, a PDE4 inhibitor, and a DPP-4 inhibitor; or v) a TGR5 receptor modulator, a GPR40 receptor modulator, a GPR119 receptor modulator, a SSTR5 receptor modulator, a PDE4 inhibitor, and a DPP-4 inhibitor. In some embodiments, the method comprises administering to the individual a TGR5 receptor modulator, a SSTR5 receptor modulator, a GPR119 receptor modulator, a PDE4 inhibitor, and a DPP-4 inhibitor. In some embodiments, the method comprises administering to the individual a TGR5 receptor modulator, a SSTR5 receptor modulator, a GPR40 receptor modulator, a PDE4 inhibitor, and a DPP-4 inhibitor. In some embodiments, the method comprises administering to the individual a TGR5 receptor modulator, a GPR40 receptor modulator, a GPR119 receptor modulator, a PDE4 inhibitor, and a DPP-4 inhibitor. In some embodiments, the method comprises administering to the individual a GPR40 receptor modulator, a GPR119 receptor modulator, a SSTR5 receptor modulator, a PDE4 inhibitor, and a DPP-4 inhibitor. In some embodiments, the method comprises administering to the individual a TGR5 receptor modulator, a GPR40 receptor modulator, a GPR119 receptor modulator, a SSTR5 receptor modulator, a PDE4 inhibitor, and a DPP-4 inhibitor.

In some embodiments, the method further comprises administering to the individual a CCK_{A} receptor modulator. In some embodiments, the CCK_{A} receptor modulator is a CCK_{A} receptor agonist.

In some embodiments, the method comprises administering to the individual: i) a TGR5 receptor modulator, a SSTR5 receptor modulator, and a CCK_{A} receptor modulator; ii) a TGR5 receptor modulator, a GPR40 receptor modulator, and a CCK_{A} receptor modulator; iii) a TGR5 receptor modulator, a GPR119 receptor modulator, and a CCK_{A} receptor modulator; iv) a GPR40 receptor modulator, a GPR119 receptor modulator, and a CCK_{A} receptor modulator; v) a GPR40 receptor modulator, a SSTR5 receptor modulator, and a CCK_{A} receptor modulator; or vi) a GPR119 receptor modulator, a SSTR5 receptor modulator, and a CCK_{A} receptor modulator. In some embodiments, the method comprises administering to the individual a TGR5 receptor modulator, a SSTR5 receptor modulator, and a CCK_{A} receptor modulator. In some embodiments, the method comprises administering to the individual a TGR5 receptor modulator, a GPR40 receptor modulator, and a CCK_{A} receptor modulator. In some embodiments, the method comprises administering to the individual a TGR5 receptor modulator, a GPR119 receptor modulator, and a CCK_{A} receptor modulator. In some embodiments, the method comprises administering to the individual a GPR40 receptor modulator, a GPR119 receptor modulator, and a CCK_{A} receptor modulator. In some embodiments, the method comprises administering to the individual a GPR40 receptor modulator, a SSTR5 receptor modulator, and a CCK_{A} receptor modulator. In some embodiments, the method comprises administering to the individual a GPR119 receptor modulator, a SSTR5 receptor modulator, and a CCK_{A} receptor modulator.

In some embodiments, the method comprises administering to the individual: i) a TGR5 receptor modulator, a SSTR5 receptor modulator, a GPR119 receptor modulator, and a CCK_{A} receptor modulator; ii) a TGR5 receptor modulator, a SSTR5 receptor modulator, a GPR40 receptor modulator, and a CCK_{A} receptor modulator; iii) a TGR5 receptor modulator, a GPR40 receptor modulator, a GPR119 receptor modulator, and a CCK_{A} receptor modulator; iv) a GPR40 receptor modulator, a GPR119 receptor modulator, a SSTR5 receptor modulator, and a CCK_{A} receptor modulator; v) or a TGR5 receptor modulator, a GPR40 receptor modulator, a GPR119 receptor modulator, a SSTR5 receptor modulator, and a CCK_{A} receptor modulator. In some embodiments, the method comprises administering to the individual a TGR5 receptor modulator, a SSTR5 receptor modulator, a GPR119 receptor modulator, and a CCK_{A} receptor modulator. In some embodiments, the method comprises administering to the individual a TGR5 receptor modulator, a SSTR5 receptor modulator, a GPR40 receptor modulator, and a CCK_{A} receptor modulator. In some embodiments, the method comprises administering to the individual a TGR5 receptor modulator, a GPR40 receptor modulator, a GPR119 receptor modulator, and a CCK_{A} receptor modulator. In some embodiments, the method comprises administering to the individual a GPR40 receptor modulator, a GPR119 receptor modulator, a SSTR5 receptor modulator, and a CCK_{A} receptor modulator. In some embodiments, the method comprises administering to the individual a TGR5 receptor modulator, a GPR40 receptor modulator, a GPR119 receptor modulator, a SSTR5 receptor modulator, and a CCK_{A} receptor modulator.

In some embodiments, the method comprises administering to the individual: i) a TGR5 receptor modulator, a SSTR5 receptor modulator, a CCK_{A} receptor modulator, and a DPP-4 inhibitor; ii) a TGR5 receptor modulator, a GPR40 receptor modulator, a CCK_{A} receptor modulator, and a DPP-4 inhibitor; iii) a TGR5 receptor modulator, a GPR119 receptor modulator, a CCK_{A} receptor modulator, and a DPP-4 inhibitor; iv) a GPR40 receptor modulator, a GPR119 receptor modulator, a CCK_{A} receptor modulator, and a DPP-4 inhibitor; v) a GPR40 receptor modulator, a SSTR5 receptor modulator, a CCK_{A} receptor modulator, and a DPP-4 inhibitor; or vi) a GPR119 receptor modulator, a SSTR5 receptor modulator, a CCK_{A} receptor modulator, and a DPP-4 inhibitor. In some embodiments, the method comprises administering to the individual: a TGR5 receptor modulator, a SSTR5 receptor modulator, a CCK_{A} receptor modulator, and a DPP-4 inhibitor. In some embodiments, the method comprises administering to the individual a TGR5 receptor modulator, a GPR40 receptor modulator, a CCK_{A} receptor modulator, and a DPP-4 inhibitor. In some embodiments, the method comprises administering to the individual a TGR5 receptor modulator, a GPR119 receptor modulator, a CCK_{A} receptor modulator, and a DPP-4 inhibitor. In some embodiments, the method comprises administering to the individual a GPR40 receptor modulator, a GPR119 receptor modulator, a CCK_{A} receptor modulator, and a DPP-4 inhibitor. In some embodiments, the method comprises administering to the individual a GPR40 receptor modulator, a SSTR5 receptor modulator, a CCK_{A} receptor modulator, and a DPP-4 inhibitor. In some embodiments, the method comprises administering to the individual a GPR119 receptor modulator, a SSTR5 receptor modulator, a CCK_{A} receptor modulator, and a DPP-4 inhibitor.

In some embodiments, the method comprises administering to the individual: i) a TGR5 receptor modulator, a SSTR5 receptor modulator, a CCK_{A} receptor modulator, and a PDE4 inhibitor; ii) a TGR5 receptor modulator, a GPR40 receptor modulator, a CCK_{A} receptor modulator, and a PDE4 inhibitor; iii) a TGR5 receptor modulator, a GPR119 receptor modulator, a CCK_{A} receptor modulator, and a PDE4 inhibitor; iv) a GPR40 receptor modulator, a GPR119 receptor modulator, a CCK_{A} receptor modulator, and a PDE4 inhibitor; v) a GPR40 receptor modulator, a SSTR5 receptor modulator, a CCK_{A} receptor modulator, and a PDE4 inhibitor; or vi) a GPR119 receptor modulator, a SSTR5 receptor modulator, a CCK_{A} receptor modulator, and a PDE4 inhibitor. In some embodiments, the method comprises administering to the individual a TGR5 receptor modulator, a SSTR5 receptor modulator, a CCK_{A} receptor modulator, and a PDE4 inhibitor. In some embodiments, the method comprises administering to the individual a TGR5 receptor modulator, a GPR40 receptor modulator, a CCK_{A} receptor modulator, and a PDE4 inhibitor. In some embodiments, the method comprises administering to the individual a TGR5 receptor modulator, a GPR119 receptor modulator, a CCK_{A} receptor modulator, and a PDE4 inhibitor. In some embodiments, the method comprises administering to the individual a GPR40 receptor modulator, a GPR119 receptor modulator, a CCK_{A} receptor modulator, and a PDE4 inhibitor. In some embodiments, the method comprises administering to the individual a GPR40 receptor modulator, a SSTR5 receptor modulator, a CCK_{A} receptor modulator, and a PDE4 inhibitor. In some embodiments, the method comprises administering to the individual a GPR119 receptor modulator, a SSTR5 receptor modulator, a CCK_{A} receptor modulator, and a PDE4 inhibitor.

In some embodiments, the method comprises administering to the individual: i) a TGR5 receptor modulator, a SSTR5 receptor modulator, a CCK_{A} receptor modulator, a PDE4 inhibitor, and a DPP-4 inhibitor; ii) a TGR5 receptor modulator, a GPR40 receptor modulator, a CCK_{A} receptor modulator, a PDE4 inhibitor, and a DPP-4 inhibitor; iii) a TGR5 receptor modulator, a GPR119 receptor modulator, a CCK_{A} receptor modulator, a PDE4 inhibitor, and a DPP-4 inhibitor; iv) a GPR40 receptor modulator, a GPR119 receptor modulator, a CCK_{A} receptor modulator, a PDE4 inhibitor, and a DPP-4 inhibitor; v) a GPR40 receptor modulator, a SSTR5 receptor modulator, a CCK_{A} receptor modulator, a PDE4 inhibitor, and a DPP-4 inhibitor; or vi) a GPR119 receptor modulator, a SSTR5 receptor modulator, a CCK_{A} receptor modulator, a PDE4 inhibitor, and a DPP-4 inhibitor. In some embodiments, the method comprises administering to the individual a TGR5 receptor modulator, a SSTR5 receptor modulator, a CCK_{A} receptor modulator, a PDE4 inhibitor, and a DPP-4 inhibitor. In some embodiments, the method comprises administering to the individual a TGR5 receptor modulator, a GPR40 receptor modulator, a CCK_{A} receptor modulator, a PDE4 inhibitor, and a DPP-4 inhibitor. In some embodiments, the method comprises administering to the individual a TGR5 receptor modulator, a GPR119 receptor modulator, a CCK_{A} receptor modulator, a PDE4 inhibitor, and a DPP-4 inhibitor. In some embodiments, the method comprises administering to the individual a GPR40 receptor modulator, a GPR119 receptor modulator, a CCK_{A} receptor modulator, a PDE4 inhibitor, and a DPP-4 inhibitor. In some embodiments, the method comprises administering to the individual a GPR40 receptor modulator, a SSTR5 receptor modulator, a CCK_{A} receptor modulator, a PDE4 inhibitor, and a DPP-4 inhibitor. In some embodiments, the method comprises administering to the individual a GPR119 receptor modulator, a SSTR5 receptor modulator, a CCK_{A} receptor modulator, a PDE4 inhibitor, and a DPP-4 inhibitor.

In some embodiments, the method comprises administering to the individual: i) a TGR5 receptor modulator, a SSTR5 receptor modulator, a GPR119 receptor modulator, a CCK_{A} receptor modulator, and a DPP-4 inhibitor; ii) a TGR5 receptor modulator, a SSTR5 receptor modulator, a GPR40 receptor modulator, a CCK_{A} receptor modulator, and a DPP-4 inhibitor; iii) a TGR5 receptor modulator, a GPR40 receptor modulator, a GPR119 receptor modulator, a CCK_{A} receptor modulator, and a DPP-4 inhibitor; iv) a GPR40 receptor modulator, a GPR119 receptor modulator, a SSTR5 receptor modulator, a CCK_{A} receptor modulator, and a DPP-4 inhibitor; or v) a TGR5 receptor modulator, a GPR40 receptor modulator, a GPR119 receptor modulator, a SSTR5 receptor modulator, a CCK_{A} receptor modulator, and a DPP-4 inhibitor. In some embodiments, the method comprises administering to the individual a TGR5 receptor modulator, a SSTR5 receptor modulator, a GPR119 receptor modulator, a CCK_{A} receptor modulator, and a DPP-4 inhibitor. In some embodiments, the method comprises administering to the individual a TGR5 receptor modulator, a SSTR5 receptor modulator, a GPR40 receptor modulator, a CCK_{A} receptor modulator, and a DPP-4 inhibitor. In some embodiments, the method comprises administering to the individual a TGR5 receptor modulator, a GPR40 receptor modulator, a GPR119 receptor modulator, a CCK_{A} receptor modulator, and a DPP-4 inhibitor. In some embodiments, the method comprises administering to the individual a GPR40 receptor modulator, a GPR119 receptor modulator, a SSTR5 receptor modulator, a CCK_{A} receptor modulator, and a DPP-4 inhibitor. In some embodiments, the method comprises administering to the individual a TGR5 receptor modulator, a GPR40 receptor modulator, a GPR119 receptor modulator, a SSTR5 receptor modulator, a CCK_{A} receptor modulator, and a DPP-4 inhibitor.

In some embodiments, the method comprises administering to the individual: i) a TGR5 receptor modulator, a SSTR5 receptor modulator, a GPR119 receptor modulator, a CCK_{A} receptor modulator, and a PDE4 inhibitor; ii) a TGR5 receptor modulator, a SSTR5 receptor modulator, a GPR40 receptor modulator, a CCK_{A} receptor modulator, and a PDE4 inhibitor; iii) a TGR5 receptor modulator, a GPR40 receptor modulator, a GPR119 receptor modulator, a CCK_{A} receptor modulator, and a PDE4 inhibitor; iv) a GPR40 receptor modulator, a GPR119 receptor modulator, a SSTR5 receptor modulator, a CCK_{A} receptor modulator, and a PDE4 inhibitor; or v) a TGR5 receptor modulator, a GPR40 receptor modulator, a GPR119 receptor modulator, a SSTR5 receptor modulator, a CCK_{A} receptor modulator, and a PDE4 inhibitor. In some embodiments, the method comprises administering to the individual a TGR5 receptor modulator, a SSTR5 receptor modulator, a GPR119 receptor modulator, a CCK_{A} receptor modulator, and a PDE4 inhibitor. In some embodiments, the method comprises administering to the individual a TGR5 receptor modulator, a SSTR5 receptor modulator, a GPR40 receptor modulator, a CCK_{A} receptor modulator, and a PDE4 inhibitor. In some embodiments, the method comprises administering to the individual a TGR5 receptor modulator, a GPR40 receptor modulator, a GPR119 receptor modulator, a CCK_{A} receptor modulator, and a PDE4 inhibitor. In some embodiments, the method comprises administering to the individual a GPR40 receptor modulator, a GPR119 receptor modulator, a SSTR5 receptor modulator, a CCK_{A} receptor modulator, and a PDE4 inhibitor. In some embodiments, the method comprises administering to the individual a TGR5 receptor modulator, a GPR40 receptor modulator, a GPR119 receptor modulator, a SSTR5 receptor modulator, a CCK_{A} receptor modulator, and a PDE4 inhibitor.

In some embodiments, the method comprises administering to the individual: i) a TGR5 receptor modulator, a SSTR5 receptor modulator, a GPR119 receptor modulator, a CCK_{A} receptor modulator, a PDE4 inhibitor, and a DPP-4 inhibitor; ii) a TGR5 receptor modulator, a SSTR5 receptor modulator, a GPR40 receptor modulator, a CCK_{A} receptor modulator, a PDE4 inhibitor, and a DPP-4 inhibitor; iii) a TGR5 receptor modulator, a GPR40 receptor modulator, a GPR119 receptor modulator, a CCK_{A} receptor modulator, a PDE4 inhibitor, and a DPP-4 inhibitor; iv) a GPR40 receptor modulator, a GPR119 receptor modulator, a SSTR5 receptor modulator, a CCK_{A} receptor modulator, a PDE4 inhibitor, and a DPP-4 inhibitor; or v) a TGR5 receptor modulator, a GPR40 receptor modulator, a GPR119 receptor modulator, a SSTR5 receptor modulator, a CCK_{A} receptor modulator, a PDE4 inhibitor, and a DPP-4 inhibitor. In some embodiments, the method comprises administering to the individual a TGR5 receptor modulator, a SSTR5 receptor modulator, a GPR119 receptor modulator, a CCK_{A} receptor modulator, a PDE4 inhibitor, and a DPP-4 inhibitor. In some embodiments, the method comprises administering to the individual a TGR5 receptor modulator, a SSTR5 receptor modulator, a GPR40 receptor modulator, a CCK_{A} receptor modulator, a PDE4 inhibitor, and a DPP-4 inhibitor. In some embodiments, the method comprises administering to the individual a TGR5 receptor modulator, a GPR40 receptor modulator, a GPR119 receptor modulator, a CCK_{A} receptor modulator, a PDE4 inhibitor, and a DPP-4 inhibitor. In some embodiments, the method comprises administering to the individual a GPR40 receptor modulator, a GPR119 receptor modulator, a SSTR5 receptor modulator, a CCK_{A} receptor modulator, a PDE4 inhibitor, and a DPP-4 inhibitor. In some embodiments, the method comprises administering to the individual a TGR5 receptor modulator, a GPR40 receptor modulator, a GPR119 receptor modulator, a SSTR5 receptor modulator, a CCK_{A} receptor modulator, a PDE4 inhibitor, and a DPP-4 inhibitor.

In some embodiments, disclosed herein is method of treating a condition or disorder involving the gut-brain axis in an individual in need thereof, the method comprising administering to the individual at least two receptor modulators selected from the group consisting of a TGR5 receptor modulator, a GPR40 receptor modulator, a GPR119 receptor modulator, a somatostatin receptor 5 (SSTR5) modulator, and a CCK_{A} receptor modulator. In some embodiments, the method comprises administering to the individual: i) a TGR5 receptor modulator and a GPR40 receptor modulator; ii) a TGR5 receptor modulator and a GPR119 receptor modulator; iii) a TGR5 receptor modulator and a SSTR5 receptor modulator; iv) a TGR5 receptor modulator and a CCK_{A} receptor modulator; v) a GPR40 receptor modulator and a GPR119 receptor modulator; vi) a GPR40 receptor modulator and a SSTR5 receptor modulator; vii) a GPR40 receptor modulator and a CCK_{A} receptor modulator; viii) a GPR119 receptor modulator and a SSTR5 receptor modulator; ix) a GPR119 receptor modulator and a CCK_{A} receptor modulator; or x) a SSTR5 receptor modulator and a CCK_{A} receptor modulator. In some embodiments, the method comprises administering to the individual at least one of the group consisting of a TGR5 receptor modulator, a GPR40 receptor modulator, and a GPR119 receptor modulator. In some embodiments, the method comprises administering to the individual: i) a TGR5 receptor modulator and a GPR40 receptor modulator; ii) a TGR5 receptor modulator and a GPR119 receptor modulator; iii) a TGR5 receptor modulator and a SSTR5 receptor modulator; iv) a TGR5 receptor modulator and a CCK_{A} receptor modulator; v) a GPR40 receptor modulator and a GPR119 receptor modulator; vi) a GPR40 receptor modulator and a SSTR5 receptor modulator; vii) a GPR40 receptor modulator and a CCK_{A} receptor modulator; viii) a GPR119 receptor modulator and a SSTR5 receptor modulator; ix) a GPR119 receptor modulator and a CCK_{A} receptor modulator. In some embodiments, the method comprises administering to the individual a TGR5 receptor modulator and a GPR40 receptor modulator. In some embodiments, the method comprises administering to the individual a TGR5 receptor modulator and a GPR119 receptor modulator. In some embodiments, the method comprises administering to the individual a TGR5 receptor modulator and a SSTR5 receptor modulator. In some embodiments, the method comprises administering to the individual a TGR5 receptor modulator and a CCK_{A} receptor modulator. In some embodiments, the method comprises administering to the individual a GPR40 receptor modulator and a GPR119 receptor modulator. In some embodiments, the method comprises administering to the individual a GPR40 receptor modulator and a SSTR5 receptor modulator. In some embodiments, the method comprises administering to the individual a GPR40 receptor modulator and a CCK_{A} receptor modulator. In some embodiments, the method comprises administering to the individual a GPR119 receptor modulator and a SSTR5 receptor modulator. In some embodiments, the method comprises administering to the individual a GPR119 receptor modulator and a CCK_{A} receptor modulator. In some embodiments, the method comprises administering to the individual a SSTR5 receptor modulator and a CCK_{A} receptor modulator. In some embodiments, at least one of the group consisting of i) the TGR5 receptor modulator, ii) the GPR40 receptor modulator, iii) the GPR119 receptor modulator, and iv) the CCK_{A} receptor modulator is a gut-restricted modulator. In some embodiments, at least one of the group consisting of i) the TGR5 receptor modulator, ii) the GPR40 receptor modulator, iii) the GPR119 receptor modulator, iv) the CCK_{A} receptor modulator, and v) the somatostatin receptor 5 (SSTR5) modulator is a gut-restricted modulator.

In some embodiments, the method comprises administering to the individual: i) a TGR5 receptor modulator and a SSTR5 receptor modulator; ii) a TGR5 receptor modulator and a GPR40 receptor modulator; iii) a TGR5 receptor modulator and a GPR119 receptor modulator; iv) a TGR5 receptor modulator and a CCK_{A} receptor modulator; v) a GPR40 receptor modulator and a GPR119 receptor modulator; vi) a GPR40 receptor modulator and a SSTR5 receptor modulator; vii) a GPR40 receptor modulator and a CCK_{A} receptor modulator; viii) a GPR119 receptor modulator and a SSTR5 receptor modulator; ix) a GPR119 receptor modulator and a CCK_{A} receptor modulator; or x) a SSTR5 receptor modulator and a CCK_{A} receptor modulator. In some embodiments, the method comprises administering to the individual: i) a TGR5 receptor modulator, a SSTR5 receptor modulator, and a GPR119 receptor modulator; ii) a TGR5 receptor modulator, a SSTR5 receptor modulator, and a GPR40 receptor modulator; iii) a TGR5 receptor modulator, a SSTR5 receptor modulator, and a CCK_{A} receptor modulator; iv) a TGR5 receptor modulator, a GPR40 receptor modulator, and a GPR119 receptor modulator; v) a TGR5 receptor modulator, a GPR40 receptor modulator, and a CCK_{A} receptor modulator; vi) a TGR5 receptor modulator, a GPR119 receptor modulator, and a CCK_{A} receptor modulator; vii) a GPR40 receptor modulator, a GPR119 receptor modulator, and a SSTR5 receptor modulator; viii) a SSTR5 receptor modulator, a GPR40 receptor modulator, and a CCK_{A} receptor modulator; ix) a SSTR5 receptor modulator, a GPR119 receptor modulator, and a CCK_{A} receptor modulator; or x) a GPR40 receptor modulator, a GPR119 receptor modulator, and a CCK_{A} receptor modulator. In some embodiments, the method comprises administering to the individual: i) a TGR5 receptor modulator, a GPR40 receptor modulator, a GPR119 receptor modulator, and a SSTR5 receptor modulator; ii) a TGR5 receptor modulator, a GPR40 receptor modulator, a GPR119 receptor modulator, and a CCK_{A} receptor modulator; iii) a TGR5 receptor modulator, a GPR40 receptor modulator, a SSTR5 receptor modulator, and a CCK_{A} receptor modulator; iv) a TGR5 receptor modulator, a GPR119 receptor modulator, a SSTR5 receptor modulator, and a CCK_{A} receptor modulator; v) a GPR40 receptor modulator, a GPR119 receptor modulator, and a SSTR5 receptor modulator, and a CCK_{A} receptor modulator; or vi) a TGR5 receptor modulator, a GPR40 receptor modulator, a GPR119 receptor modulator, a SSTR5 receptor modulator, and a CCK_{A} receptor modulator.

In some embodiments, the method comprises administering to the individual: i) a TGR5 receptor modulator and a GPR40 receptor modulator; ii) a TGR5 receptor modulator and a GPR119 receptor modulator; or iii) a GPR40 receptor modulator and a GPR119 receptor modulator. In some embodiments, the method comprises administering to the individual a TGR5 receptor modulator, a GPR40 receptor modulator, and a GPR119 receptor modulator. In some embodiments, the method further comprises administering to the individual a SSTR5 receptor modulator. In some embodiments, the method further comprises administering to the individual a CCK_{A} receptor modulator.

In some embodiments, the method further comprises administering to the individual a PDE4 inhibitor. In some embodiments, the method comprises administering to the individual: i) a TGR5 receptor modulator, a SSTR5 receptor modulator, and a PDE4 inhibitor; ii) a TGR5 receptor modulator, a GPR40 receptor modulator, and a PDE4 inhibitor; iii) a TGR5 receptor modulator, a GPR119 receptor modulator, and a PDE4 inhibitor; iv) a TGR5 receptor modulator, a CCK_{A} receptor modulator, and a PDE4 inhibitor; v) a GPR40 receptor modulator, a GPR119 receptor modulator, and a PDE4 inhibitor; vi) a GPR40 receptor modulator, a SSTR5 receptor modulator, and a PDE4 inhibitor; vii) a GPR40 receptor modulator, a CCK_{A} receptor modulator, and a PDE4 inhibitor; viii) a GPR119 receptor modulator, a SSTR5 receptor modulator, and a PDE4 inhibitor; ix) a GPR119 receptor modulator, a CCK_{A} receptor modulator, and a PDE4 inhibitor; or x) a SSTR5 receptor modulator, a CCK_{A} receptor modulator, and a PDE4 inhibitor. In some embodiments, the method comprises administering to the individual: i) a TGR5 receptor modulator, a SSTR5 receptor modulator, a GPR119 receptor modulator, and a PDE4 inhibitor; ii) a TGR5 receptor modulator, a SSTR5 receptor modulator, a GPR40 receptor modulator, and a PDE4 inhibitor; iii) a TGR5 receptor modulator, a SSTR5 receptor modulator, a CCK_{A} receptor modulator, and a PDE4 inhibitor; iv) a TGR5 receptor modulator, a GPR40 receptor modulator, a GPR119 receptor modulator, and a PDE4 inhibitor; v) a TGR5 receptor modulator, a GPR40 receptor modulator, a CCK_{A} receptor modulator, and a PDE4 inhibitor; vi) a TGR5 receptor modulator, a GPR119 receptor modulator, a CCK_{A} receptor modulator, and a PDE4 inhibitor; vii) a GPR40 receptor modulator, a GPR119 receptor modulator, a SSTR5 receptor modulator, and a PDE4 inhibitor; viii) a SSTR5 receptor modulator, a GPR40 receptor modulator, a CCK_{A} receptor modulator, and a PDE4 inhibitor; ix) a SSTR5 receptor modulator, a GPR119 receptor modulator, a CCK_{A} receptor modulator, and a PDE4 inhibitor; or x) a GPR40 receptor modulator, a GPR119 receptor modulator, a CCK_{A} receptor modulator, and a PDE4 inhibitor. In some embodiments, the method comprises administering to the individual: i) a TGR5 receptor modulator, a GPR40 receptor modulator, a GPR119 receptor modulator, a SSTR5 receptor modulator, and a PDE4 inhibitor; ii) a TGR5 receptor modulator, a GPR40 receptor modulator, a GPR119 receptor modulator, a CCK_{A} receptor modulator, and a PDE4 inhibitor; iii) a TGR5 receptor modulator, a GPR40 receptor modulator, a SSTR5 receptor modulator, a CCK_{A} receptor modulator, and a PDE4 inhibitor; iv) a TGR5 receptor modulator, a GPR119 receptor modulator, a SSTR5 receptor modulator, a CCK_{A} receptor modulator, and a PDE4 inhibitor; v) a GPR40 receptor modulator, a GPR119 receptor modulator, a SSTR5 receptor modulator, a CCK_{A} receptor modulator, and a PDE4 inhibitor; or vi) a TGR5 receptor modulator, a GPR40 receptor modulator, a GPR119 receptor modulator, a SSTR5 receptor modulator, a CCK_{A} receptor modulator, and a PDE4 inhibitor.

In some embodiments, the method further comprises administering to the individual a DPP-4 inhibitor. In some embodiments, the method comprises administering to the individual: i) a TGR5 receptor modulator, a SSTR5 receptor modulator, and a DPP-4 inhibitor; ii) a TGR5 receptor modulator, a GPR40 receptor modulator, and a DPP-4 inhibitor; iii) a TGR5 receptor modulator, a GPR119 receptor modulator, and a DPP-4 inhibitor; iv) a TGR5 receptor modulator, a CCK_{A} receptor modulator, and a DPP-4 inhibitor; v) a GPR40 receptor modulator, a GPR119 receptor modulator, and a DPP-4 inhibitor; vi) a GPR40 receptor modulator, a SSTR5 receptor modulator, and a DPP-4 inhibitor; vii) a GPR40 receptor modulator, a CCK_{A} receptor modulator, and a DPP-4 inhibitor; viii) a GPR119 receptor modulator, a SSTR5 receptor modulator, and a DPP-4 inhibitor; ix) a GPR119 receptor modulator, a CCK_{A} receptor modulator, and a DPP-4 inhibitor; or x) a SSTR5 receptor modulator, a CCK_{A} receptor modulator, and a DPP-4 inhibitor. In some embodiments, the method comprises administering to the individual: i) a TGR5 receptor modulator, a SSTR5 receptor modulator, a GPR119 receptor modulator, and a DPP-4 inhibitor; ii) a TGR5 receptor modulator, a SSTR5 receptor modulator, a GPR40 receptor modulator, and a DPP-4 inhibitor; iii) a TGR5 receptor modulator, a SSTR5 receptor modulator, a CCK_{A} receptor modulator, and a DPP-4 inhibitor; iv) a TGR5 receptor modulator, a GPR40 receptor modulator, a GPR119 receptor modulator, and a DPP-4 inhibitor; v) a TGR5 receptor modulator, a GPR40 receptor modulator, a CCK_{A} receptor modulator, and a DPP-4 inhibitor; vi) a TGR5 receptor modulator, a GPR119 receptor modulator, a CCK_{A} receptor modulator, and a DPP-4 inhibitor; vii) a GPR40 receptor modulator, a GPR119 receptor modulator, a SSTR5 receptor modulator, and a DPP-4 inhibitor; viii) a SSTR5 receptor modulator, a GPR40 receptor modulator, a CCK_{A} receptor modulator, and a DPP-4 inhibitor; ix) a SSTR5 receptor modulator, a GPR119 receptor modulator, a CCK_{A} receptor modulator, and a DPP-4 inhibitor; or x) a GPR40 receptor modulator, a GPR119 receptor modulator, a CCK_{A} receptor modulator, and a DPP-4 inhibitor. In some embodiments, the method comprises administering to the individual: i) a TGR5 receptor modulator, a GPR40 receptor modulator, a GPR119 receptor modulator, a SSTR5 receptor modulator, and a DPP-4 inhibitor; ii) a TGR5 receptor modulator, a GPR40 receptor modulator, a GPR119 receptor modulator, a CCK_{A} receptor modulator, and a DPP-4 inhibitor; iii) a TGR5 receptor modulator, a GPR40 receptor modulator, a SSTR5 receptor modulator, a CCK_{A} receptor modulator, and a DPP-4 inhibitor; iv) a TGR5 receptor modulator, a GPR119 receptor modulator, a SSTR5 receptor modulator, a CCK_{A} receptor modulator, and a DPP-4 inhibitor; v) a GPR40 receptor modulator, a GPR119 receptor modulator, a SSTR5 receptor modulator, a CCK_{A} receptor modulator, and a DPP-4 inhibitor; or vi) a TGR5 receptor modulator, a GPR40 receptor modulator, a GPR119 receptor modulator, a SSTR5 receptor modulator, a CCK_{A} receptor modulator, and a DPP-4 inhibitor. In some embodiments, the method further comprises administering to the individual a PDE4 inhibitor. In some embodiments, the method comprises administering to the individual: i) a TGR5 receptor modulator, a SSTR5 receptor modulator, a PDE4 inhibitor, and a DPP-4 inhibitor; ii) a TGR5 receptor modulator, a GPR40 receptor modulator, a PDE4 inhibitor, and a DPP-4 inhibitor; iii) a TGR5 receptor modulator, a GPR119 receptor modulator, a PDE4 inhibitor, and a DPP-4 inhibitor; iv) a TGR5 receptor modulator, a CCK_{A} receptor modulator, a PDE4 inhibitor, and a DPP-4 inhibitor; v) a GPR40 receptor modulator, a GPR119 receptor modulator, a PDE4 inhibitor, and a DPP-4 inhibitor; vi) a GPR40 receptor modulator, a SSTR5 receptor modulator, a PDE4 inhibitor, and a DPP-4 inhibitor; vii) a GPR40 receptor modulator, a CCK_{A} receptor modulator, a PDE4 inhibitor, and a DPP-4 inhibitor; viii) a GPR119 receptor modulator, a SSTR5 receptor modulator, a PDE4 inhibitor, and a DPP-4 inhibitor; ix) a GPR119 receptor modulator, a CCK_{A} receptor modulator, a PDE4 inhibitor, and a DPP-4 inhibitor; or x) a SSTR5 receptor modulator, a CCK_{A} receptor modulator, a PDE4 inhibitor, and a DPP-4 inhibitor. In some embodiments, the method comprises administering to the individual: i) a TGR5 receptor modulator, a SSTR5 receptor modulator, a GPR119 receptor modulator, a PDE4 inhibitor, and a DPP-4 inhibitor; ii) a TGR5 receptor modulator, a SSTR5 receptor modulator, a GPR40 receptor modulator, a PDE4 inhibitor, and a DPP-4 inhibitor; iii) a TGR5 receptor modulator, a SSTR5 receptor modulator, a CCK_{A} receptor modulator, a PDE4 inhibitor, and a DPP-4 inhibitor; iv) a TGR5 receptor modulator, a GPR40 receptor modulator, a GPR119 receptor modulator, a PDE4 inhibitor, and a DPP-4 inhibitor; v) a TGR5 receptor modulator, a GPR40 receptor modulator, a CCK_{A} receptor modulator, a PDE4 inhibitor, and a DPP-4 inhibitor; vi) a TGR5 receptor modulator, a GPR119 receptor modulator, a CCK_{A} receptor modulator, a PDE4 inhibitor, and a DPP-4 inhibitor; vii) a GPR40 receptor modulator, a GPR119 receptor modulator, a SSTR5 receptor modulator, a PDE4 inhibitor, and a DPP-4 inhibitor; viii) a SSTR5 receptor modulator, a GPR40 receptor modulator, a CCK_{A} receptor modulator, a PDE4 inhibitor, and a DPP-4 inhibitor; ix) a SSTR5 receptor modulator, a GPR119 receptor modulator, a CCK_{A} receptor modulator, a PDE4 inhibitor, and a DPP-4 inhibitor; or x) a GPR40 receptor modulator, a GPR119 receptor modulator, a CCK_{A} receptor modulator, a PDE4 inhibitor, and a DPP-4 inhibitor. In some embodiments, the method comprises administering to the individual: i) a TGR5 receptor modulator, a GPR40 receptor modulator, a GPR119 receptor modulator, a SSTR5 receptor modulator, a PDE4 inhibitor, and a DPP-4 inhibitor; ii) a TGR5 receptor modulator, a GPR40 receptor modulator, a GPR119 receptor modulator, a CCK_{A} receptor modulator, a PDE4 inhibitor, and a DPP-4 inhibitor; iii) a TGR5 receptor modulator, a GPR40 receptor modulator, a SSTR5 receptor modulator, a CCK_{A} receptor modulator, a PDE4 inhibitor, and a DPP-4 inhibitor; iv) a TGR5 receptor modulator, a GPR119 receptor modulator, a SSTR5 receptor modulator, a CCK_{A} receptor modulator, a PDE4 inhibitor, and a DPP-4 inhibitor; v) a GPR40 receptor modulator, a GPR119 receptor modulator, a SSTR5 receptor modulator, a CCK_{A} receptor modulator, a PDE4 inhibitor, and a DPP-4 inhibitor; or vi) a TGR5 receptor modulator, a GPR40 receptor modulator, a GPR119 receptor modulator, a SSTR5 receptor modulator, a CCK_{A} receptor modulator, a PDE4 inhibitor, and a DPP-4 inhibitor.

In some embodiments, the condition is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, celiac disease, and enteritis, including chemotherapy-induced enteritis or radiation-induced enteritis; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis. In some embodiments, the condition is a metabolic disorder. In some embodiments, the metabolic disorder is diabetes. In other embodiments, the metabolic disorder is obesity. In some embodiments, the condition involving the gut-brain axis is a nutritional disorder. In some embodiments, the nutritional disorder is short bowel syndrome, intestinal failure, or intestinal insufficiency. In some embodiments, the nutritional disorder is short bowel syndrome.

In some embodiments of the methods described herein, the combination of at least two receptor modulators selected from the group consisting of a TGR5 receptor modulator, a GPR40 receptor modulator, a GPR119 receptor modulator, and a somatostatin receptor 5 (SSTR5) modulator increases gut hormone release. In some embodiments, the combination increases peptide hormone release. In some embodiments, the combination increases GLP-1, GIP, PYY, CCK, or a combination thereof. In some embodiments, the combination decreases food intake in the individual. In some embodiments, the combination decreases daily food intake in the individual. In some embodiments the combination increases gut growth.

In some embodiments, modulating the activity of multiple receptors simultaneously as described herein results in elevated hormone levels. In some embodiments, modulating the activity of multiple receptors simultaneously as described herein results in synergistically elevated hormone levels. In some embodiments, modulating the activity of multiple receptors simultaneously as described herein results in elevated hormone secretion. In some embodiments, modulating the activity of multiple receptors simultaneously as described herein results in synergistically elevated hormone secretion. In some embodiments, modulating the activity of multiple receptors simultaneously as described herein results in hormone levels higher than those when modulating the activity of any single receptor. In some embodiments, modulating the activity of multiple receptors simultaneously as described herein results in hormone secretion higher than that when modulating the activity of any single receptor. In some embodiments, modulating the activity of multiple receptors simultaneously as described herein elicits a greater biological response, for example, increased insulin secretion, lower food consumption, increased body mass reduction, increased cAMP levels, increased nutrient absorption, increased small intestinal length, increased small intestinal weight, increased villus height, or increased villus height/crypt depth ratio than when modulating the activity of any single receptor. In some embodiments, modulating the activity multiple receptors simultaneously as described herein is preferred for the methods described herein relative to modulating the activity of a single receptor.

### Gut-Restricted Modulators

In some embodiments, the receptor modulators are gut-restricted. In some embodiments, the receptor modulators are designed to be substantially non-permeable or substantially non-bioavailable in the blood stream. In some embodiments, the receptor modulators are designed to modulate receptor activity in the gut, for example, stimulate hormone secretion, but are themselves substantially non-systemic.

In some embodiments, a gut-restricted modulator has low oral bioavailability. In some embodiments, a gut-restricted modulator has <10% oral bioavailability, <8% oral bioavailability, <5% oral bioavailability, <3% oral bioavailability, or <2% oral bioavailability.

In some embodiments, a gut-restricted agonist, gut-restricted antagonist, or gut-restricted inverse agonist has low oral bioavailability. In some embodiments, a gut-restricted agonist, gut-restricted antagonist, or gut-restricted inverse agonist has <10% oral bioavailability, <8% oral bioavailability, <5% oral bioavailability, <3% oral bioavailability, or <2% oral bioavailability.

In some embodiments, the unbound plasma levels of a gut-restricted modulator are lower than the IC₅₀ value or the EC₅₀ value of the gut-restricted modulator against its receptor. In some embodiments, the unbound plasma levels of a gut-restricted modulator are significantly lower than the IC₅₀ value or the EC₅₀ value of the gut-restricted modulator against its receptor. In some embodiments, the unbound plasma levels of a gut-restricted modulator are 2-fold, 10-fold, 20-fold, 30-fold, 40-fold, 50-fold, or 100-fold lower than the IC₅₀ value or the EC₅₀ value of the gut-restricted modulator against its receptor.

In some embodiments, the unbound plasma levels of a gut-restricted agonist, gut-restricted antagonist, or gut-restricted inverse agonist are lower than the IC₅₀ value or the EC₅₀ value of the gut-restricted modulator against its receptor. In some embodiments, the unbound plasma levels of a gut-restricted agonist, gut-restricted antagonist, or gut-restricted inverse agonist are significantly lower than the IC₅₀ value or the EC₅₀ value of the gut-restricted modulator against its receptor. In some embodiments, the unbound plasma levels of a gut-restricted agonist, gut-restricted antagonist, or gut-restricted inverse agonist are 2-fold, 10-fold, 20-fold, 30-fold, 40-fold, 50-fold, or 100-fold lower than the IC₅₀ value or the EC₅₀ value of the gut-restricted agonist, gut-restricted antagonist, or gut-restricted inverse agonist against its receptor.

In some embodiments, a gut-restricted modulator has low systemic exposure. In some embodiments, the systemic exposure of a gut-restricted modulator is, for example, less than 500, less than 200, less than 100, less than 50, less than 20, less than 10, or less than 5 nM, bound or unbound, in blood serum. In some embodiments, the systemic exposure of a gut-restricted modulator is, for example, less than 500, less than 200, less than 100, less than 50, less than 20, less than 10, or less than 5 ng/mL, bound or unbound, in blood serum.

In some embodiments, a gut-restricted agonist, gut-restricted antagonist, or gut-restricted inverse agonist has low systemic exposure. In some embodiments, the systemic exposure of a gut-restricted agonist, gut-restricted antagonist, or gut-restricted inverse agonist is, for example, less than 500, less than 200, less than 100, less than 50, less than 20, less than 10, or less than 5 nM, bound or unbound, in blood serum. In some embodiments, the systemic exposure of a gut-restricted agonist, gut-restricted antagonist, or gut-restricted inverse agonist is, for example, less than 500, less than 200, less than 100, less than 50, less than 20, less than 10, or less than 5 ng/mL, bound or unbound, in blood serum.

In some embodiments, a gut-restricted modulator has high intestinal exposure. In some embodiments, the intestinal exposure of a gut-restricted modulator is, for example, greater than 1000, 5000, 10000, 50000, 100000, or 500000 nM.

In some embodiments, a gut-restricted agonist, gut-restricted antagonist, or gut-restricted inverse agonist has high intestinal exposure. In some embodiments, the intestinal exposure of a gut-restricted agonist, gut-restricted antagonist, or gut-restricted inverse agonist is, for example, greater than 1000, 5000, 10000, 50000, 100000, or 500000 nM.

In some embodiments, a gut-restricted modulator has low permeability. In some embodiments, a gut-restricted modulator has low intestinal permeability. In some embodiments, the permeability of a gut-restricted modulator is, for example, less than 5.0×10⁻⁶ cm/s, less than 2.0×10⁻⁶ cm/s, less than 1.5×10⁻⁶ cm/s, less than 1.0×10⁻⁶ cm/s, less than 0.75×10⁻⁶ cm/s, less than 0.50×10⁻⁶ cm/s, less than 0.25×10⁻⁶ cm/s, less than 0.10×10⁻⁶ cm/s, or less than 0.05×10⁻⁶ cm/s.

In some embodiments, a gut-restricted agonist, gut-restricted antagonist, or gut-restricted inverse agonist has low permeability. In some embodiments, a gut-restricted agonist, gut-restricted antagonist, or gut-restricted inverse agonist has low intestinal permeability. In some embodiments, the permeability of a gut-restricted agonist, gut-restricted antagonist, or gut-restricted inverse agonist is, for example, less than 5.0×10⁻⁶ cm/s, less than 2.0×10⁻⁶ cm/s, less than 1.5×10⁻⁶ cm/s, less than 1.0×10⁻⁶ cm/s, less than 0.75×10⁻⁶ cm/s, less than 0.50×10⁻⁶ cm/s, less than 0.25×10⁻⁶ cm/s, less than 0.10×10⁻⁶ cm/s, or less than 0.05×10⁻⁶ cm/s.

In some embodiments, a gut-restricted modulator has low absorption. In some embodiments, the absorption of a gut-restricted modulator is less than less than 20%, or less than 10%, less than 5%, or less than 1%.

In some embodiments, a gut-restricted agonist, gut-restricted antagonist, or gut-restricted inverse agonist has low absorption. In some embodiments, the absorption of a gut-restricted agonist, gut-restricted antagonist, or gut-restricted inverse agonist is less than 20%, or less than 10%, less than 5%, or less than 1%.

In some embodiments, a gut-restricted modulator has high plasma clearance. In some embodiments, a gut-restricted modulator is undetectable in plasma in less than 8 hours, less than 6 hours, less than 4 hours, less than 3 hours, less than 120 min, less than 90 min, less than 60 min, less than 45 min, less than 30 min, or less than 15 min.

In some embodiments, a gut-restricted agonist, gut-restricted antagonist, or gut-restricted inverse agonist has high plasma clearance. In some embodiments, a gut-restricted agonist, gut-restricted antagonist, or gut-restricted inverse agonist is undetectable in plasma in less than 8 hours, less than 6 hours, less than 4 hours, less than 3 hours, less than 120 min, less than 90 min, less than 60 min, less than 45 min, less than 30 min, or less than 15 min.

In some embodiments, a gut-restricted modulator is rapidly metabolized upon administration. In some embodiments, a gut-restricted modulator has a short half-life. In some embodiments, the half-life of a gut-restricted modulator is less than less than 8 hours, less than 6 hours, less than 4 hours, less than 3 hours, less than 120 min, less than 90 min, less than 60 min, less than 45 min, less than 30 min, or less than 15 min. In some embodiments, the metabolites of a gut-restricted modulator have rapid clearance. In some embodiments, the metabolites of a gut-restricted modulator are undetectable in less than 8 hours, less than 6 hours, less than 4 hours, less than 3 hours, less than 120 min, less than 90 min, less than 60 min, less than 45 min, less than 30 min, or less than 15 min. In some embodiments, the metabolites of a gut-restricted modulator have low bioactivity. In some embodiments, the IC₅₀ value or the EC₅₀ value of the metabolites of a gut-restricted modulator is 10-fold, 20-fold, 30-fold, 40-fold, 50-fold, 100-fold, 500-fold, or 1000-fold higher than the IC₅₀ value or the EC₅₀ value of the gut-restricted modulator against its receptor. In some embodiments, the metabolites of a gut-restricted modulator have rapid clearance and low bioactivity.

In some embodiments, a gut-restricted agonist, gut-restricted antagonist, or gut-restricted inverse agonist is rapidly metabolized upon administration. In some embodiments, a gut-restricted agonist, gut-restricted antagonist, or gut-restricted inverse agonist has a short half-life. In some embodiments, the half-life of a gut-restricted agonist, gut-restricted antagonist, or gut-restricted inverse agonist is less than 8 hours, less than 6 hours, less than 4 hours, less than 3 hours, less than 120 min, less than 90 min, less than 60 min, less than 45 min, less than 30 min, or less than 15 min. In some embodiments, the metabolites of a gut-restricted agonist, gut-restricted antagonist, or gut-restricted inverse agonist have rapid clearance. In some embodiments, the metabolites of a gut-restricted agonist, gut-restricted antagonist, or gut-restricted inverse agonist are undetectable in less than 8 hours, less than 6 hours, less than 4 hours, less than 3 hours, less than 120 min, less than 90 min, less than 60 min, less than 45 min, less than 30 min, or less than 15 min. In some embodiments, the metabolites of a gut-restricted agonist, gut-restricted antagonist, or gut-restricted inverse agonist have low bioactivity. In some embodiments, the IC₅₀ value or the EC₅₀ value of the metabolites of a gut-restricted agonist, gut-restricted antagonist, or gut-restricted inverse agonist is 10-fold, 20-fold, 30-fold, 40-fold, 50-fold, 100-fold, 500-fold, or 1000-fold higher than the IC₅₀ value or the EC₅₀ value of the gut-restricted agonist, gut-restricted antagonist, or gut-restricted inverse agonist against its receptor. In some embodiments, the metabolites of a gut-restricted agonist, gut-restricted antagonist, or gut-restricted inverse agonist have rapid clearance and low bioactivity.

In some embodiments of the methods described herein, the TGR5 receptor modulator is gut-restricted. In some embodiments, the TGR5 receptor modulator is a gut-restricted TGR5 agonist. In some embodiments, the TGR5 modulator is covalently bonded to a kinetophore. In some embodiments, the TGR5 modulator is covalently bonded to a kinetophore through a linker.

In some embodiments of the methods described herein, the GPR40 receptor modulator is gut-restricted. In some embodiments, the GPR40 receptor modulator is a gut-restricted GPR40 agonist. In some embodiments, the GPR40 modulator is covalently bonded to a kinetophore. In some embodiments, the GPR40 modulator is covalently bonded to a kinetophore through a linker.

In some embodiments of the methods described herein, the GPR119 receptor modulator is gut-restricted. In some embodiments, the GPR119 receptor modulator is a gut-restricted GPR119 agonist. In some embodiments, the GPR119 modulator is covalently bonded to a kinetophore. In some embodiments, the GPR119 modulator is covalently bonded to a kinetophore through a linker.

In some embodiments of the methods described herein, the SSTR5 receptor modulator is gut-restricted. In some embodiments, the SSTR5 receptor modulator is a gut-restricted SSTR5 antagonist. In some embodiments, the SSTR5 modulator is covalently bonded to a kinetophore. In some embodiments, the SSTR5 modulator is covalently bonded to a kinetophore through a linker.

In some embodiments of the methods described herein, at least two receptor modulators are covalently bonded to each other. In some embodiments, the at least two receptor modulators are covalently bonded to each other. In some embodiments, the at least two receptor modulators are covalently bonded to each other through a linker. In some embodiments, at least two receptor modulators are covalently bonded, optionally through a linker. In some embodiments, the at least two receptor modulators which are covalently bonded to each other are gut-restricted.

In some embodiments of the methods described herein, a receptor modulator is covalently bonded to at least one other receptor modulator. In some embodiments of the methods described herein, a receptor modulator is covalently bonded to at least two other receptor modulators. In some embodiments of the methods described herein, a receptor modulator is covalently bonded to at least three other receptor modulators. In some embodiments, the receptor modulator is covalently bonded to the at least one other receptor modulator. In some embodiments, the receptor modulator is covalently bonded to the at least one other receptor modulator through a linker. In some embodiments, the receptor modulator is covalently bonded to the at least one other receptor modulator, optionally through a linker. In some embodiments, the receptor modulators which are covalently bonded to each other are gut-restricted.

In some embodiments of the methods described herein, at least two receptor modulators selected from the group consisting of a gut-restricted agonist, a gut-restricted antagonist, and a gut-restricted inverse agonist are covalently bonded to each other. In some embodiments, the at least two receptor modulators are covalently bonded to each other. In some embodiments, the at least two receptor modulators are covalently bonded to each other through a linker. In some embodiments, at least two receptor modulators are covalently bonded, optionally through a linker. In some embodiments, the at least two receptor modulators which are covalently bonded to each other are gut-restricted.

In some embodiments of the methods described herein, a receptor modulator selected from the group consisting of a gut-restricted agonist, a gut-restricted antagonist, and a gut-restricted inverse agonist is covalently bonded to at least one other receptor modulator selected from the group consisting of a gut-restricted agonist, a gut-restricted antagonist, and a gut-restricted inverse agonist. In some embodiments of the methods described herein, a receptor modulator is covalently bonded to at least two other receptor modulators selected from the group consisting of a gut-restricted agonist, a gut-restricted antagonist, and a gut-restricted inverse agonist. In some embodiments of the methods described herein, a receptor modulator is covalently bonded to at least three other receptor modulators selected from the group consisting of a gut-restricted agonist, a gut-restricted antagonist, and a gut-restricted inverse agonist. In some embodiments, the receptor modulator is covalently bonded to the at least one other receptor modulator. In some embodiments, the receptor modulator is covalently bonded to the at least one other receptor modulator through a linker. In some embodiments, the receptor modulator is covalently bonded to the at least one other receptor modulator, optionally through a linker. In some embodiments, the receptor modulators which are covalently bonded to each other are gut-restricted.

In some embodiments of the methods described herein, the at least two receptor modulators which are covalently bonded to each other are selected from the group consisting of a TGR5 receptor modulator, a GPR40 receptor modulator, a GPR119 receptor modulator, and a somatostatin receptor 5 (SSTR5) modulator. In some embodiments, the at least two receptor modulators which are covalently bonded to each other are selected from the group consisting of a TGR5 agonist, a GPR40 agonist, a GPR119 agonist, and a somatostatin receptor 5 (SSTR5) antagonist.

### Pharmaceutical Compositions

In some embodiments, disclosed herein is a pharmaceutical composition comprising at least two receptor modulators selected from the group consisting of a TGR5 receptor modulator, a GPR40 receptor modulator, a GPR119 receptor modulator, and a somatostatin receptor 5 (SSTR5) modulator. In some embodiments, the pharmaceutical composition comprises: i) a TGR5 receptor modulator and a SSTR5 receptor modulator; ii) a TGR5 receptor modulator and a GPR40 receptor modulator; iii) a TGR5 receptor modulator and a GPR119 receptor modulator; iv) a GPR40 receptor modulator and a GPR119 receptor modulator; v) a GPR40 receptor modulator and a SSTR5 receptor modulator; vi) a GPR119 receptor modulator and a SSTR5 receptor modulator; vii) a TGR5 receptor modulator, a SSTR5 receptor modulator, and a GPR119 receptor modulator; viii) a TGR5 receptor modulator, a SSTR5 receptor modulator, and a GPR40 receptor modulator; ix) a TGR5 receptor modulator, a GPR40 receptor modulator, and a GPR119 receptor modulator; x) a GPR40 receptor modulator, a GPR119 receptor modulator, and a SSTR5 receptor modulator; or xi) a TGR5 receptor modulator, a GPR40 receptor modulator, a GPR119 receptor modulator, and a SSTR5 receptor modulator. In some embodiments the at least two receptor modulators are covalently bonded, optionally through a linker.

In some embodiments, disclosed herein is a pharmaceutical composition comprising at least two receptor modulators selected from the group consisting of a TGR5 receptor modulator, a GPR40 receptor modulator, a GPR119 receptor modulator, a somatostatin receptor 5 (SSTR5) modulator, and a CCK_{A} receptor modulator. In some embodiments, the pharmaceutical composition comprises: i) a TGR5 receptor modulator and a SSTR5 receptor modulator; ii) a TGR5 receptor modulator and a GPR40 receptor modulator; iii) a TGR5 receptor modulator and a GPR119 receptor modulator; iv) a TGR5 receptor modulator and a CCK_{A} receptor modulator; v) a GPR40 receptor modulator and a GPR119 receptor modulator; vi) a GPR40 receptor modulator and a SSTR5 receptor modulator; vii) a GPR40 receptor modulator and a CCK_{A} receptor modulator; viii) a GPR119 receptor modulator and a SSTR5 receptor modulator; ix) a GPR119 receptor modulator and a CCK_{A} receptor modulator; x) a SSTR5 receptor modulator and a CCK_{A} receptor modulator; xi) a TGR5 receptor modulator, a SSTR5 receptor modulator, and a GPR119 receptor modulator; xii) a TGR5 receptor modulator, a SSTR5 receptor modulator, and a GPR40 receptor modulator; xiii) a TGR5 receptor modulator, a SSTR5 receptor modulator, and a CCK_{A} receptor modulator; xiv) a TGR5 receptor modulator, a GPR40 receptor modulator, and a GPR119 receptor modulator; xv) a TGR5 receptor modulator, a GPR40 receptor modulator, and a CCK_{A} receptor modulator; xvi) a TGR5 receptor modulator, a GPR119 receptor modulator, and a CCK_{A} receptor modulator; xvii) a GPR40 receptor modulator, a GPR119 receptor modulator, and a SSTR5 receptor modulator; xviii) a SSTR5 receptor modulator, a GPR40 receptor modulator, and a CCK_{A} receptor modulator; xix) a SSTR5 receptor modulator, a GPR119 receptor modulator, and a CCK_{A} receptor modulator; xx) a GPR40 receptor modulator, a GPR119 receptor modulator, and a CCK_{A} receptor modulator; xxi) a TGR5 receptor modulator, a GPR40 receptor modulator, a GPR119 receptor modulator, and a SSTR5 receptor modulator; xxii) a TGR5 receptor modulator, a GPR40 receptor modulator, a GPR119 receptor modulator, and a CCK_{A} receptor modulator; xxiii) a TGR5 receptor modulator, a GPR40 receptor modulator, a SSTR5 receptor modulator, and a CCK_{A} receptor modulator; xxiv) a TGR5 receptor modulator, a GPR119 receptor modulator, a SSTR5 receptor modulator, and a CCK_{A} receptor modulator; xxv) a GPR40 receptor modulator, a GPR119 receptor modulator, and a SSTR5 receptor modulator, and a CCK_{A} receptor modulator; or xxvi) a TGR5 receptor modulator, a GPR40 receptor modulator, a GPR119 receptor modulator, a SSTR5 receptor modulator, and a CCK_{A} receptor modulator. In some embodiments the at least two receptor modulators are covalently bonded, optionally through a linker.

In some embodiments, the modulators are combined with a pharmaceutically suitable (or acceptable) carrier (also referred to herein as a pharmaceutically suitable (or acceptable) excipient, physiologically suitable (or acceptable) excipient, or physiologically suitable (or acceptable) carrier) selected on the basis of a chosen route of administration, e.g., oral administration, and standard pharmaceutical practice.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants.

### EXAMPLES

### Example 1: Organoid Model of Gut Hormone Release

Murine gut organoids in Complete Crypt Culture Media (CCCM; see Sato T, Clevers H. Methods Mol Biol 2013; 945:319-28 DOI: 10.1007/978-1-62703-125-7_19) were plated at a density of typically 40-50 organoids per well in a 96-well plate. Five days after passage, CCCM was removed from the wells of the organoid plate, and organoids were washed with 0.125 mL/well of Hank's Balance Salt Solution [−] Calcium Chloride [−] Magnesium Chloride [−] Magnesium Sulfate (HBSS). HBSS was removed by aspiration, and 0.075 mL of basal culture media (BCM: Advanced DMEM/F12, 2 mM Glutamax, 10 mM HEPES, 100 U/mL penicillin, 0.100 mg/mL streptomycin) at 37° C. was added to each well. The plate was incubated at 37° C. for 90 minutes. Secretion media was removed from organoids and placed into a PCR plate containing Halt Protease Inhibitor Cocktail (0.001 mL; Thermo Scientific, Prod #78438), DPP-4 Inhibitor (0.002 mL; EMD Millipore, cat. no. DPP4-010). This was the baseline sample set. A 0.075 mL aliquot of test agent in BCM/0.1% DMSO at 37° C. was added to each well. The plate was incubated at 37° C. for 150 minutes. Secretion media was removed from the organoids and placed into a PCR plate containing Halt Protease Inhibitor Cocktail (0.001 mL; Thermo Scientific, Prod #78438), DPP-4 Inhibitor (0.002 mL; EMD Millipore, cat. no. DPP4-010) and 0.001 mL of 0.5 M EDTA. The medium was mixed and immediately stored at −80° C. This was the stimulated secretion sample set. Gut peptides were measured as outlined below, and data were reported as fold increase of stimulated secretion sample over baseline sample.

### Measurement of Gut Peptides

Gut hormones were measured according to known procedures. Total GLP-1, active GLP-1, and total PYY were measured using Meso Scale Discovery (MSD) assay technology employing commercially available assay kits and following the published protocol. GIP was measured on a Clarion plate reader using a Millipore GIP assay kit and following the published protocol. CCK was measured on a Clarion plate reader using a Phoenix Pharmaceuticals assay kit and following the published protocol.

Table 1 demonstrates the fold increase of gut peptides after treatment with modulators with exemplary mechanisms of action.

TABLE 1

| Study | | Concentration | Gut | Hormone Fold-Increase | | | |
|---|---|---|---|---|---|---|---|
| # | Mechanism(s)^{a} | (μM)^{b} | Segment^{c} | GIP^{d} | GLP-1^{d} | PYY^{d} | CCK^{d} |
| 1-1 | GPR40 | 10 | proximal | | 9.7 | | |
| 1-1 | TGR5 | 10 | proximal | | 7.8 | | |
| 1-2 | GPR40 | 10 | jejunal | 2.9 | 5.4 | | |
| 1-2 | TGR5 | 10 | jejunal | 5.3 | 13.5 | | |
| 1-2 | GPR119 | 10 | jejunal | 1.4 | 4.3 | | |
| 1-2 | GPR40/GPR119 | 10/10 | jejunal | 5.8 | 11.5 | | |
| 1-2 | TGR5/GPR119 | 10/10 | jejunal | 5.9 | 10.5 | | |
| 1-2 | GPR40/TGR5/GPR119 | 10/10/10 | jejunal | 8.0 | 16.3 | | |
| 1-2 | GPR40 | 10 | ileal | 2.1 | 7.1 | 4.2 | |
| 1-2 | TGR5 | 10 | ileal | 5.1 | 18.4 | 11.2 | |
| 1-2 | GPR119 | 10 | ileal | 1.9 | 3.4 | 3.2 | |
| 1-2 | GPR40/GPR119 | 10/10 | ileal | 15.0 | 13.6 | 6.6 | |
| 1-2 | TGR5/GPR119 | 10/10 | ileal | 11.5 | 12.6 | 10.0 | |
| 1-2 | GPR40/TGR5/GPR119 | 10/10/10 | ileal | 5.5 | 22.2 | 15.4 | |
| 1-3 | GPR40/GPR119 | 10/10 | jejunal | | 33.9 | | |
| 1-3 | GPR40/GPR119 | 1/1 | jejunal | | 17.1 | | |
| 1-4 | GPR40 | 10 | duodenum | | | | 0.4 |
| 1-4 | GPR119 | 10 | duodenum | | | | 0.3 |
| 1-4 | GPR40/GPR119 | 10/10 | duodenum | | | | 0.8 |
| 1-4 | GPR40 | 10 | duodenum | | | | 3.0 |
| 1-4 | TGR5 | 10 | duodenum | | | | 4.6 |
| 1-4 | GPR119 | 10 | duodenum | | | | 2.6 |
| 1-4 | GPR40/GPR119 | 10/10 | duodenum | | | | 5.1 |
| 1-4 | TGR5/GPR119 | 10/10 | duodenum | | | | 6.6 |
| 1-4 | GPR40 | 10 | jejunal | | | | 1.9 |
| 1-4 | TGR5 | 10 | jejunal | | | | 2.7 |
| 1-4 | GPR119 | 10 | jejunal | | | | 0.9 |
| 1-4 | GPR40/TGR5 | 10/10 | jejunal | | | | 3.3 |
| 1-4 | GPR40/GPR119 | 10/10 | jejunal | | | | 2.4 |
| 1-4 | TGR5/GPR119 | 10/10 | jejunal | | | | 2.6 |
| 1-5 | GPR40 | 10 | jejunal | | 6.7 | | |
| 1-5 | GPR119 | 10 | jejunal | | 3.4 | | |
| 1-5 | GPR40/GPR119 | 10/10 | jejunal | | 12.7 | | |
| 1-6 | GPR40 | 10 | jejunal | | 6.1 | | |
| 1-6 | GPR119 | 10 | jejunal | | 2.3 | | |
| 1-6 | GPR40/GPR119 | 10/10 | jejunal | | 9.8 | | |
| 1-7 | GPR40 | 10 | duodenum | | | | 2.4 |
| 1-7 | GPR119 | 10 | duodenum | | | | 1.5 |
| 1-7 | GPR40/GPR119 | 10/10 | duodenum | | | | 4.7 |
| 1-8 | SSTR5 | 10 | ileal | | 1.6 pg/mL | | |
| 1-8 | TGR5 | 1 | ileal | | 11 pg/mL | | |
| 1-8 | SSTR5/TGR5 | 10/1 | Heal | | 15.3 pg/mL | | |
| 1-9 | GPR40 | 10 | jejunal | | 5.3 | | |
| 1-9 | GPR119 | 10 | jejunal | | 1.8 | | |
| 1-9 | GPR40/GPR119 | 10/10 | jejunal | | 10.2 | | |
| 1-10 | GPR40 | 10 | duodenum | | 1.7 | | |
| 1-10 | GPR119 | 10 | duodenum | | 1.1 | | |
| 1-10 | GPR40/GPR119 | 10/10 | duodenum | | 2.5 | | |
| 1-11 | GPR40 | 1 | jejunal | | 5.2 | | |
| 1-11 | TGR5 | 1 | jejunal | | 5.4 | | |
| 1-11 | GPR40/TGR5 | 1/1 | jejunal | | 15.4 | | |
| 1-12 | GPR40 | 10 | jejunal | | 1.6 | | |
| 1-12 | GPR119 | 10 | jejunal | | 2.3 | | |
| 1-12 | GPR40/GPR119 | 10/10 | jejunal | | 2.5 | | |
| 1-12 | TGR5 | 0.1 | ileal | | 12.5 | | |
| 1-12 | SSTR5 | 10 | ileal | | 3.8 | | |
| 1-12 | SSTR5/TGR5 | 10/0.1 | ileal | | 25.5 | | |

^{a}GPR40 = treatment with GPR40 agonist; GPR119 = treatment with GPR119 agonist, TGR5 = treatment with TGR5 agonist; SSTR5 = treatment with SSTR5 antagonist;
^{b}concentration of each agonist/antagonist;
^{c}organoid gut segment;
^{d}fold increase of stimulated secretion sample over baseline sample, except where otherwise indicated.

As demonstrated in Table 1, combination treatments lead to greater increases in hormone secretion than the treatments with single mechanisms of action.

### Example 2: Ex Vivo Gut Peptide Secretion in Mice

Male C57BL/6J mice 10-12 weeks old were fasted overnight in clean cages. On the day of study, mice were anesthetized with ketamine plus xylazine. An incision was made in the abdomen of each animal to expose the intestine. Test article or vehicle was injected in specific location(s) in the intestine: duodenum, jejunal/ileal junction or colon (or a combination thereof) based on the expression of the target. Blood was collected via cardiac puncture 30 min post dose for measurement of gut peptides.

Table 2 shows plasma levels of gut peptides after treatment with vehicle or modulator(s) with exemplary mechanisms of action.

TABLE 2

| Study | | Dose | Gut | Plasma Levels (pg/mL) | | |
|---|---|---|---|---|---|---|
| # | Mechanism(s)^{a} | (mg/kg) | Segment^{b} | GIP | GLP-1 | PYY |
| 2-1 | vehicle | | J/I + C | | 3.0 | 78.6 |
| 2-1 | GPR40 | 30 | J/I + C | | 29.3 | 113.6 |
| 2-1 | GPR119 | 20 | J/I + C | | 34.6 | 173.7 |
| 2-1 | GPR40/GPR119 | 30/20 | J/I + C | | 71.4 | 318.9 |
| 2-2 | vehicle | | J/I | | 5.6 | |
| 2-2 | GPR40 | 30 | J/I | | 18.9 | |
| 2-2 | GPR40/GPR119 | 30/30 | J/I | | 67.5 | |
| 2-3 | vehicle | | J/I | | 13.3 | 39.7 |
| 2-3 | GPR40 | 30 | J/I | | 24.2 | 89.2 |
| 2-3 | GPR40/GPR119 | 30/30 | J/I | | 79.1 | 158.2 |
| 2-4 | vehicle | | J/I | 56.1 | 5.9 | |
| 2-4 | GPR119 gut restricted | 10 | J/I | 107.6 | 25.6 | |
| 2-4 | GPR119/SSTR5 | 10/30 | J/I | 145.1 | 30 | |
| 2-4 | GPR40/GPR119 | 30/20 | J/I | 94.7 | 53.7 | |
| 2-4 | GPR40/GPR119/SSTR5 | 30/20/30 | J/I | 130.2 | 125.5 | |
| 2-5 | vehicle | | C | | 27.7 | |
| 2-5 | TGR5 soft drug | 30 | C | | 74 | |
| 2-5 | TGR5/SSTR5 | 30/3 | C | | 276.1 | |
| 2-6 | Vehicle | | J/I | 144.5 | 10.8 | 33.2 |
| 2-6 | GPR119 | 20 | J/I | 180.4 | 25.9 | 65.8 |
| 2-6 | PDE4 | 30 | J/I | 181.6 | 23.4 | 85.4 |
| 2-6 | GPR119/PDE4 | 20/30 | J/I | 352.1 | 46.4 | 161.8 |

^{a}GPR40 = treatment with GPR40 agonist; GPR119 = treatment with GPR119 agonist, TGR5 = treatment with TGR5 agonist; SSTR5 = treatment with SSTR5 antagonist; PDE4 = treatment with PDE4 inhibitor;
^{b}J/I = jejunal/ileal junction, C = colon.

### Example 3: In Vivo Gut Peptide Secretion in Mice

Male C57BL/6J mice 10-12 weeks old were acclimated to dosing (e.g., oral gavage) 2-3 times prior to the study. On the day of the study, food was removed for 5-6 hours, then the mice were dosed with test article or vehicle (e.g., by oral gavage at a volume of 10 mL/kg). Animals were euthanized with carbon dioxide typically 30 min post dose. Blood was collected via cardiac puncture for measurement of gut peptides.

As demonstrated in Table 3, GLP-1 release is 5.6-fold higher than baseline when mice are treated with both a TGR5 agonist and a GPR40 agonist, which is significantly higher than with a TGR5 agonist (3.5-fold) or a GPR40 agonist (2.8-fold) alone.

TABLE 3

| | | Dose | Plasma Levels (pg/mL) | |
|---|---|---|---|---|
| Study # | Mechanism(s)^{a} | (mg/kg) | GIP | GLP-1 |
| 3-1 | vehicle | | 151.7 | 20.1 |
| 3-1 | TGR5 | 50 | 230.3 | 50.2 |
| 3-1 | GPR40 | 10 | 854.9 | 56.9 |
| 3-1 | TGR5 + GPR40 | 50/10 | 1401.1 | 112.2 |

^{a}GPR40 = treatment with GPR40 agonist;
TGR5 = treatment with TGR5 agonist.

### Example 4: 2-Hour Food Intake in Mice

Male C57BL/6J mice ˜8 weeks old were singly housed in a reverse light cycle room 2 weeks prior to study. During this run-in period, mice were dosed by oral gavage three times to acclimate them to the procedure, and they were provided high fat diet (Research Diets D12492i) for 2 hours weekly. One day before the study mice were fasted for 5 hours prior to lights out and dosed with vehicle 30 min prior to lights out. They were given access to high fat diet at lights out, and 2-h dark cycle food intake was measured as baseline. The animals were randomized to groups based on 2-h dark cycle food intake (primary) and body weight (secondary). On the day of the study, mice were fasted for 5 hours and then dosed orally with test article or vehicle 30 min prior to lights out. After lights out, the mice were immediately given access to high fat diet. Food intake was measured at 2 h.

Table 4 shows food intake after treatment with modulators with exemplary mechanisms of action.

TABLE 4

| | | | Food |
|---|---|---|---|
| | | Dose | Intake |
| Study # | Mechanism(s)^{a} | (mg/kg)^{b} | (g) |
| 3-1 | vehicle | | 1.2 |
| 3-1 | GPR40 | 30 | 1.1 |
| 3-1 | GPR119 | 20 | 1.0 |
| 3-1 | GPR40/GPR119 | 30/20 | 0.89 |
| 3-1 | GPR40/GPR119/DPP-4 | 30/20/10 | 0.97 |
| 3-2 | vehicle | | 1.3 |
| 3-2 | SSTR5 | 30 | 1.3 |
| 3-2 | GPR40/GPR119 | 30/20 | 0.94 |
| 3-2 | GPR40/GPR119/SSTR5 | 30/20/30 | 0.39 |
| 3-3 | vehicle | | 1.3 |
| 3-3 | GPR40/GPR119 | 30/20 | 1.3 |
| 3-3 | GPR40/GPR119/SSTR5 | 30/20/30 | 0.86 |
| 3-3 | GPR40/GPR119/DPP-4 | 30/20/10 | 1.2 |
| 3-3 | GPR40/GPR119/SSTR5/DPP-4 | 30/20/30/10 | 0.64 |
| 3-4 | vehicle | | 1.5 |
| 3-4 | GPR40/TGR5 | 30/50 | 0.62 |
| 3-4 | GPR40/SSTR5 | 30/30 | 0.71 |
| 3-4 | TGR5/SSTR5 | 50/30 | 0.42 |
| 3-4 | GPR40/T GR5/SSTR5 | 30/50/30 | 0.11 |
| 3-5 | vehicle | | 1.5 |
| 3-5 | GPR40/TGR5 | 30/50 | 0.68 |
| 3-5 | GPR40/DPP-4 | 30/10 | 1.3 |
| 3-5 | TGR5/DPP-4 | 50/10 | 0.81 |
| 3-5 | GPR40/TGR5/DPP-4 | 30/50/10 | 0.51 |
| 3-6 | vehicle | | 1.5 |
| 3-6 | TGR5 | 50 | 0.82 |
| 3-6 | GPR40/TGR5 | 30/50 | 0.87 |
| 3-6 | GPR40/TGR5/SSTR5 | 30/50/30 | 0.52 |
| 3-6 | GPR40/TGR5/DPP-4 | 30/50/10 | 0.68 |
| 3-8 | vehicle | | 1.2 |
| 3-8 | GPR40/TGR5 | 30/50 | 0.45 |
| 3-8 | SSTR5/DPP-4 | 30/10 | 0.95 |
| 3-8 | GPR40/SSTR5/DPP-4 | 30/30/10 | 0.35 |
| 3-8 | TGR5/SSTR5/DPP-4 | 50/30/10 | 0.24 |
| 3-9 | vehicle | | 1.5 |
| 3-9 | GPR40/GPR119 | 30/30 | 1.4 |
| 3-9 | GPR40/GPR119/SSTR5 | 30/30/30 | 1.1 |
| 3-9 | GPR40/GPR119/DPP-4 | 30/30/10 | 1.4 |
| 3-9 | GPR40/GPR119/SSTR5/DPP-4 | 30/30/30/10 | 0.96 |

^{a}GPR40 = treatment with GPR40 agonist;
GPR119 = treatment with GPR119 agonist,
TGR5 = treatment with TGR5 agonist;
SSTR5 = treatment with SSTR5 antagonist;
DPP-4 = treatment with DPP-4 inhibitor;
^{b}dose of each agonist/antagonist.

### Example 5. 10-Day Weight Loss in eDIO Mice

Single-housed male C57Bl/6J established diet-induced obese (eDIO) mice were acclimated to oral gavage multiple times prior to the study. On the day of the study (Day 0), mice (18-20 weeks old) were sorted into groups based on body weight and maintained on 60% high fat diet. Test article or vehicle was administered just prior to lights out each day for 7 or 10 days. Food intake was monitored each afternoon (24-h feeding behavior). Food from the day before was discarded, and fresh food was provided daily. Body weight was taken in the afternoon prior to dosing. Approximately 18 h after the final dose, ambient glucose was measured, and the animal was euthanized. Blood was collected for measurement of gut peptides.

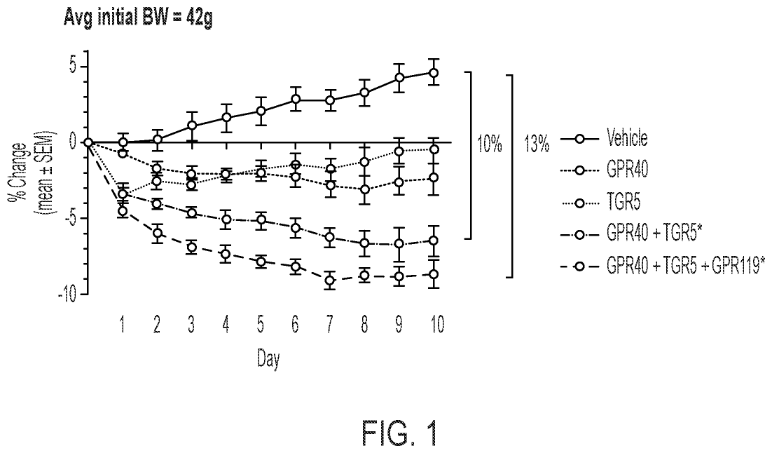

Table 5 and FIG. 1 show body weight reduction after treatment with modulators with exemplary mechanisms of action.

TABLE 5

| | | | Body Weight | |
|---|---|---|---|---|
| Study | | Dose | (% change | # |
| # | Mechanism(s)^{a} | (mg/kg)^{b} | from vehicle) | Days |
| 4−1 | GPR40 | 30 | −7.1 | 10 |
| 4−1 | TGR5 | 50 | −5.3 | 10 |
| 4−1 | GPR40/TGR5 | 30/50 | −11.2 | 10 |
| 4−1 | GPR40/TGR5/GPR119 | 30/50/20 | −13.4 | 10 |
| 4−2 | GPR40/GPR119 | 30/20 | −2.7 | 10 |
| 4−2 | GPR40/GPR119/SSTR5 | 30/20/30 | 0.4 | 10 |
| 4−2 | GPR40/GPR119/DPP-4 | 30/20/10 | −2.3 | 10 |
| 4−2 | GPR40/GPR119/SSTR5/DPP-4 | 30/20/30/10 | −6.4 | 10 |
| 4−3 | GPR40/TGR5 | 30/50 | −9.9 | 7 |
| 4−3 | SSTR5/DPP-4 | 30/10 | −1.7 | 7 |
| 4−3 | GPR40/GPR119/SSTR5/DPP-4 | 30/20/30/10 | −8.8 | 7 |

^{a}GPR40 = treatment with GPR40 agonist;
GPR119 = treatment with GPR119 agonist,
TGR5 = treatment with TGR5 agonist;
SSTR5 = treatment with SSTR5 antagonist;
DPP-4 = treatment with DPP-4 inhibitor;
^{b}dose of each agonist/antagonist.

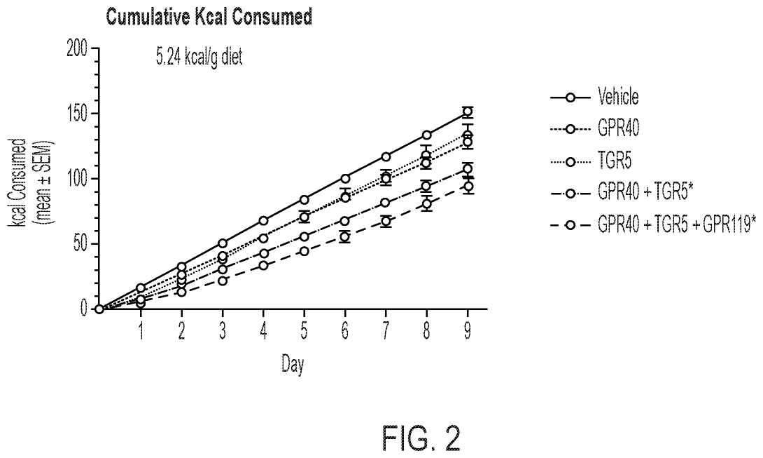

Also, as demonstrated in FIG. 2, mice with diet induced obesity that are treated with the described combinations of a TGR5 agonist, GPR40 agonist, GPR119 agonist, SSTR antagonist, and/or DPP-4 inhibitor show a marked decrease in total food intake.

### Example 6: 16-Hour Food Intake and Body Weight in Mice

Male C57BL/6J mice ˜8 weeks old were singly housed in a normal light cycle room 2 weeks prior to study. During this run-in period, mice were dosed by oral gavage three times to acclimate them to the procedure, and body weight was monitored two-to-three times per week. Mice losing >1 g of body weight were not included in the study cohort. Once during the acclimation period, mice were fasted for 5 h, dosed with vehicle, then provided with high fat diet (Research Diets D12492i) during the light cycle, and food intake was measured for 16 hours to provide a food intake baseline. Mice were randomized based on food intake baseline (primary) and body weight (secondary). On the day of the study, mice were fasted for 5 hours and then dosed orally with test article or vehicle 30 min prior to lights out. After lights out, the mice were immediately given access to high fat diet. Food intake and body weight was measured at 16 h.

Table 6 shows food intake and body weight and food intake after treatment with a gut-restricted CCK_{A} agonist alone and in combination with a gut-restricted GPR119 agonist with and without a gut-restricted SSTR5 antagonist.

TABLE 6

| Study | | Dose | Food Intake | Body Weight |
|---|---|---|---|---|
| # | Mechanism(s)^{a} | (mg/kg)^{b} | (g) | Change (g) |
| 6-1 | vehicle | | 3.8 | +1.1 |
| 6-1 | CCK_{A} | 3 | 3.3 | +0.55 |
| 6-1 | CCK_{A}/GPR119 | 3/100 | 3.2 | +0.43 |
| 6-1 | CCK_{A}/GPR119/SSTR5 | 3/100/30 | 2.7 | +0.34 |

^{a}CCK_{A} = treatment with CCK_{A} agonist;
GPR119 = treatment with GPR119 agonist,
SSTR5 = treatment with SSTR5 antagonist;
^{b}dose of each agonist/antagonist.

We claim:

1. A method of treating a condition or disorder involving the gut-brain axis in an individual in need thereof, the method comprising administering to the individual at least two receptor modulators selected from the group consisting of a TGR5 receptor modulator, a GPR40 receptor modulator, a GPR119 receptor modulator, a somatostatin receptor 5 (SSTR5) modulator, and a CCK_{A} receptor modulator; wherein at least one of the group consisting of i) the TGR5 receptor modulator, ii) the GPR40 receptor modulator, iii) the GPR119 receptor modulator, iv) the somatostatin receptor 5 (SSTR5) modulator, and v) the CCK_{A} receptor modulator is a gut-restricted modulator.

2. The method of claim 1, the method comprising administering to the individual:
i) a TGR5 receptor modulator and a SSTR5 receptor modulator;
ii) a TGR5 receptor modulator and a GPR40 receptor modulator;
iii) a TGR5 receptor modulator and a GPR119 receptor modulator;
iv) a TGR5 receptor modulator and a CCK_{A} receptor modulator;
v) a GPR40 receptor modulator and a GPR119 receptor modulator;
vi) a GPR40 receptor modulator and a SSTR5 receptor modulator;
vii) a GPR40 receptor modulator and a CCK_{A} receptor modulator;
viii) a GPR119 receptor modulator and a SSTR5 receptor modulator;
ix) a GPR119 receptor modulator and a CCK_{A} receptor modulator; or
x) a SSTR5 receptor modulator and a CCK_{A} receptor modulator.

3. The method of claim 1, the method comprising administering to the individual:
i) a TGR5 receptor modulator, a SSTR5 receptor modulator, and a GPR119 receptor modulator;
ii) a TGR5 receptor modulator, a SSTR5 receptor modulator, and a GPR40 receptor modulator;
iii) a TGR5 receptor modulator, a SSTR5 receptor modulator, and a CCK_{A} receptor modulator;
iv) a TGR5 receptor modulator, a GPR40 receptor modulator, and a GPR119 receptor modulator;
v) a TGR5 receptor modulator, a GPR40 receptor modulator, and a CCK_{A} receptor modulator;
vi) a TGR5 receptor modulator, a GPR119 receptor modulator, and a CCK_{A} receptor modulator;
vii) a GPR40 receptor modulator, a GPR119 receptor modulator, and a SSTR5 receptor modulator;
viii) a SSTR5 receptor modulator, a GPR40 receptor modulator, and a CCK_{A} receptor modulator;
ix) a SSTR5 receptor modulator, a GPR119 receptor modulator, and a CCK_{A} receptor modulator; or
x) a GPR40 receptor modulator, a GPR119 receptor modulator, and a CCK_{A} receptor modulator.

4. The method of claim 1, the method comprising administering to the individual:
i) a TGR5 receptor modulator, a GPR40 receptor modulator, a GPR119 receptor modulator, and a SSTR5 receptor modulator;
ii) a TGR5 receptor modulator, a GPR40 receptor modulator, a GPR119 receptor modulator, and a CCK_{A} receptor modulator;
iii) a TGR5 receptor modulator, a GPR40 receptor modulator, a SSTR5 receptor modulator, and a CCK_{A} receptor modulator;
iv) a TGR5 receptor modulator, a GPR119 receptor modulator, a SSTR5 receptor modulator, and a CCK_{A} receptor modulator;
v) a GPR40 receptor modulator, a GPR119 receptor modulator, and a SSTR5 receptor modulator, and a CCK_{A} receptor modulator; or
vi) a TGR5 receptor modulator, a GPR40 receptor modulator, a GPR119 receptor modulator, a SSTR5 receptor modulator, and a CCK_{A} receptor modulator.

5. The method of claim 1, the method comprising administering to the individual:
i) a TGR5 receptor modulator and a GPR40 receptor modulator;
ii) a TGR5 receptor modulator and a GPR119 receptor modulator; or
iii) a GPR40 receptor modulator and a GPR119 receptor modulator.

6. The method of claim 1, the method comprising administering to the individual a TGR5 receptor modulator, a GPR40 receptor modulator, and a GPR119 receptor modulator.

7. The method of claim 5 or claim 6, further comprising administering to the individual a SSTR5 receptor modulator.

8. The method of any one of claims 5-7, further comprising administering to the individual a CCK_{A} receptor modulator.

9. The method of any one of the preceding claims, further comprising administering to the individual a PDE4 inhibitor.

10. The method of claim 9, the method comprising administering to the individual:
i) a TGR5 receptor modulator, a SSTR5 receptor modulator, and a PDE4 inhibitor;
ii) a TGR5 receptor modulator, a GPR40 receptor modulator, and a PDE4 inhibitor;
iii) a TGR5 receptor modulator, a GPR119 receptor modulator, and a PDE4 inhibitor;
iv) a TGR5 receptor modulator, a CCK_{A} receptor modulator, and a PDE4 inhibitor;
v) a GPR40 receptor modulator, a GPR119 receptor modulator, and a PDE4 inhibitor;
vi) a GPR40 receptor modulator, a SSTR5 receptor modulator, and a PDE4 inhibitor;
vii) a GPR40 receptor modulator, a CCK_{A} receptor modulator, and a PDE4 inhibitor;
viii) a GPR119 receptor modulator, a SSTR5 receptor modulator, and a PDE4 inhibitor;
ix) a GPR119 receptor modulator, a CCK_{A} receptor modulator, and a PDE4 inhibitor; or
x) a SSTR5 receptor modulator, a CCK_{A} receptor modulator, and a PDE4 inhibitor.

11. The method of claim 9, the method comprising administering to the individual:
i) a TGR5 receptor modulator, a SSTR5 receptor modulator, a GPR119 receptor modulator, and a PDE4 inhibitor;
ii) a TGR5 receptor modulator, a SSTR5 receptor modulator, a GPR40 receptor modulator, and a PDE4 inhibitor;
iii) a TGR5 receptor modulator, a SSTR5 receptor modulator, a CCK_{A} receptor modulator, and a PDE4 inhibitor;
iv) a TGR5 receptor modulator, a GPR40 receptor modulator, a GPR119 receptor modulator, and a PDE4 inhibitor;
v) a TGR5 receptor modulator, a GPR40 receptor modulator, a CCK_{A} receptor modulator, and a PDE4 inhibitor;
vi) a TGR5 receptor modulator, a GPR119 receptor modulator, a CCK_{A} receptor modulator, and a PDE4 inhibitor;
vii) a GPR40 receptor modulator, a GPR119 receptor modulator, a SSTR5 receptor modulator, and a PDE4 inhibitor;
viii) a SSTR5 receptor modulator, a GPR40 receptor modulator, a CCK_{A} receptor modulator, and a PDE4 inhibitor;
ix) a SSTR5 receptor modulator, a GPR119 receptor modulator, a CCK_{A} receptor modulator, and a PDE4 inhibitor; or
x) a GPR40 receptor modulator, a GPR119 receptor modulator, a CCK_{A} receptor modulator, and a PDE4 inhibitor.

12. The method of claim 9, the method comprising administering to the individual:
i) a TGR5 receptor modulator, a GPR40 receptor modulator, a GPR119 receptor modulator, a SSTR5 receptor modulator, and a PDE4 inhibitor;
ii) a TGR5 receptor modulator, a GPR40 receptor modulator, a GPR119 receptor modulator, a CCK_{A} receptor modulator, and a PDE4 inhibitor;
iii) a TGR5 receptor modulator, a GPR40 receptor modulator, a SSTR5 receptor modulator, a CCK_{A} receptor modulator, and a PDE4 inhibitor;
iv) a TGR5 receptor modulator, a GPR119 receptor modulator, a SSTR5 receptor modulator, a CCK_{A} receptor modulator, and a PDE4 inhibitor;
v) a GPR40 receptor modulator, a GPR119 receptor modulator, and a SSTR5 receptor modulator, a CCK_{A} receptor modulator, and a PDE4 inhibitor; or
vi) a TGR5 receptor modulator, a GPR40 receptor modulator, a GPR119 receptor modulator, a SSTR5 receptor modulator, a CCK_{A} receptor modulator, and a PDE4 inhibitor.

13. The method of any one of the preceding claims, further comprising administering to the individual a DPP-4 inhibitor.

14. The method of claim 13, the method comprising administering to the individual:
i) a TGR5 receptor modulator, a SSTR5 receptor modulator, and a DPP-4 inhibitor;
ii) a TGR5 receptor modulator, a GPR40 receptor modulator, and a DPP-4 inhibitor;
iii) a TGR5 receptor modulator, a GPR119 receptor modulator, and a DPP-4 inhibitor;
iv) a TGR5 receptor modulator, a CCK_{A} receptor modulator, and a DPP-4 inhibitor;
v) a GPR40 receptor modulator, a GPR119 receptor modulator, and a DPP-4 inhibitor;
vi) a GPR40 receptor modulator, a SSTR5 receptor modulator, and a DPP-4 inhibitor;
vii) a GPR40 receptor modulator, a CCK_{A} receptor modulator, and a DPP-4 inhibitor;
viii) a GPR119 receptor modulator, a SSTR5 receptor modulator, and a DPP-4 inhibitor;
ix) a GPR119 receptor modulator, a CCK_{A} receptor modulator, and a DPP-4 inhibitor; or
x) a SSTR5 receptor modulator, a CCK_{A} receptor modulator, and a DPP-4 inhibitor.

15. The method of claim 13, the method comprising administering to the individual:
i) a TGR5 receptor modulator, a SSTR5 receptor modulator, a GPR119 receptor modulator, and a DPP-4 inhibitor;
ii) a TGR5 receptor modulator, a SSTR5 receptor modulator, a GPR40 receptor modulator, and a DPP-4 inhibitor;
iii) a TGR5 receptor modulator, a SSTR5 receptor modulator, a CCK_{A} receptor modulator, and a DPP-4 inhibitor;
iv) a TGR5 receptor modulator, a GPR40 receptor modulator, a GPR119 receptor modulator, and a DPP-4 inhibitor;
v) a TGR5 receptor modulator, a GPR40 receptor modulator, a CCK_{A} receptor modulator, and a DPP-4 inhibitor;
vi) a TGR5 receptor modulator, a GPR119 receptor modulator, a CCK_{A} receptor modulator, and a DPP-4 inhibitor;
vii) a GPR40 receptor modulator, a GPR119 receptor modulator, a SSTR5 receptor modulator, and a DPP-4 inhibitor;
viii) a SSTR5 receptor modulator, a GPR40 receptor modulator, a CCK_{A} receptor modulator, and a DPP-4 inhibitor;
ix) a SSTR5 receptor modulator, a GPR119 receptor modulator, a CCK_{A} receptor modulator, and a DPP-4 inhibitor; or
x) a GPR40 receptor modulator, a GPR119 receptor modulator, a CCK_{A} receptor modulator, and a DPP-4 inhibitor.

16. The method of claim 13, the method comprising administering to the individual:
i) a TGR5 receptor modulator, a GPR40 receptor modulator, a GPR119 receptor modulator, a SSTR5 receptor modulator, and a DPP-4 inhibitor;
ii) a TGR5 receptor modulator, a GPR40 receptor modulator, a GPR119 receptor modulator, a CCK_{A} receptor modulator, and a DPP-4 inhibitor;
iii) a TGR5 receptor modulator, a GPR40 receptor modulator, a SSTR5 receptor modulator, a CCK_{A} receptor modulator, and a DPP-4 inhibitor;
iv) a TGR5 receptor modulator, a GPR119 receptor modulator, a SSTR5 receptor modulator, a CCK_{A} receptor modulator, and a DPP-4 inhibitor;
v) a GPR40 receptor modulator, a GPR119 receptor modulator, and a SSTR5 receptor modulator, a CCK_{A} receptor modulator, and a DPP-4 inhibitor; or
vi) a TGR5 receptor modulator, a GPR40 receptor modulator, a GPR119 receptor modulator, a SSTR5 receptor modulator, a CCK_{A} receptor modulator, and a DPP-4 inhibitor.

17. The method of claim 13, the method comprising administering to the individual:
i) a TGR5 receptor modulator, a SSTR5 receptor modulator, a PDE4 inhibitor, and a DPP-4 inhibitor;
ii) a TGR5 receptor modulator, a GPR40 receptor modulator, a PDE4 inhibitor, and a DPP-4 inhibitor;
iii) a TGR5 receptor modulator, a GPR119 receptor modulator, a PDE4 inhibitor, and a DPP-4 inhibitor;
iv) a TGR5 receptor modulator, a CCK_{A} receptor modulator, a PDE4 inhibitor, and a DPP-4 inhibitor;
v) a GPR40 receptor modulator, a GPR119 receptor modulator, a PDE4 inhibitor, and a DPP-4 inhibitor;
vi) a GPR40 receptor modulator, a SSTR5 receptor modulator, a PDE4 inhibitor, and a DPP-4 inhibitor;
vii) a GPR40 receptor modulator, a CCK_{A} receptor modulator, a PDE4 inhibitor, and a DPP-4 inhibitor;
viii) a GPR119 receptor modulator, a SSTR5 receptor modulator, a PDE4 inhibitor, and a DPP-4 inhibitor;
ix) a GPR119 receptor modulator, a CCK_{A} receptor modulator, a PDE4 inhibitor, and a DPP-4 inhibitor; or
x) a SSTR5 receptor modulator, a CCK_{A} receptor modulator, a PDE4 inhibitor, and a DPP-4 inhibitor.

18. The method of claim 13, the method comprising administering to the individual:
i) a TGR5 receptor modulator, a SSTR5 receptor modulator, a GPR119 receptor modulator, a PDE4 inhibitor, and a DPP-4 inhibitor;
ii) a TGR5 receptor modulator, a SSTR5 receptor modulator, a GPR40 receptor modulator, a PDE4 inhibitor, and a DPP-4 inhibitor;
iii) a TGR5 receptor modulator, a SSTR5 receptor modulator, a CCK_{A} receptor modulator, a PDE4 inhibitor, and a DPP-4 inhibitor;
iv) a TGR5 receptor modulator, a GPR40 receptor modulator, a GPR119 receptor modulator, a PDE4 inhibitor, and a DPP-4 inhibitor;
v) a TGR5 receptor modulator, a GPR40 receptor modulator, a CCK_{A} receptor modulator, a PDE4 inhibitor, and a DPP-4 inhibitor;
vi) a TGR5 receptor modulator, a GPR119 receptor modulator, a CCK_{A} receptor modulator, a PDE4 inhibitor, and a DPP-4 inhibitor;
vii) a GPR40 receptor modulator, a GPR119 receptor modulator, a SSTR5 receptor modulator, a PDE4 inhibitor, and a DPP-4 inhibitor;
viii) a SSTR5 receptor modulator, a GPR40 receptor modulator, a CCK_{A} receptor modulator, a PDE4 inhibitor, and a DPP-4 inhibitor;
ix) a SSTR5 receptor modulator, a GPR119 receptor modulator, a CCK_{A} receptor modulator, a PDE4 inhibitor, and a DPP-4 inhibitor; or
x) a GPR40 receptor modulator, a GPR119 receptor modulator, a CCK_{A} receptor modulator, a PDE4 inhibitor, and a DPP-4 inhibitor.

19. The method of claim 13, the method comprising administering to the individual:
i) a TGR5 receptor modulator, a GPR40 receptor modulator, a GPR119 receptor modulator, a SSTR5 receptor modulator, a PDE4 inhibitor, and a DPP-4 inhibitor;
ii) a TGR5 receptor modulator, a GPR40 receptor modulator, a GPR119 receptor modulator, a CCK_{A} receptor modulator, a PDE4 inhibitor, and a DPP-4 inhibitor;
iii) a TGR5 receptor modulator, a GPR40 receptor modulator, a SSTR5 receptor modulator, a CCK_{A} receptor modulator, a PDE4 inhibitor, and a DPP-4 inhibitor;
iv) a TGR5 receptor modulator, a GPR119 receptor modulator, a SSTR5 receptor modulator, a CCK_{A} receptor modulator, a PDE4 inhibitor, and a DPP-4 inhibitor;
v) a GPR40 receptor modulator, a GPR119 receptor modulator, and a SSTR5 receptor modulator, a CCK_{A} receptor modulator, a PDE4 inhibitor, and a DPP-4 inhibitor; or
vi) a TGR5 receptor modulator, a GPR40 receptor modulator, a GPR119 receptor modulator, a SSTR5 receptor modulator, a CCK_{A} receptor modulator, a PDE4 inhibitor, and a DPP-4 inhibitor.

20. The method of any one of the preceding claims, wherein the TGR5 receptor modulator, the GPR40 receptor modulator, and GPR119 receptor modulator are gut-restricted modulators.

21. The method of any one of the preceding claims, wherein the TGR5 receptor modulator is a gut-restricted agonist of a TGR receptor.

22. The method of any one of the preceding claims, wherein the GPR40 receptor modulator is a gut-restricted agonist of a GPR40 receptor.

23. The method of any one of the preceding claims, wherein the GPR119 receptor modulator is a gut-restricted agonist of a GPR119 receptor.

24. The method of any one of the preceding claims, wherein the SSTR5 receptor modulator is a gut-restricted antagonist of a SSTR5 receptor.

25. The method of any one of the preceding claims, wherein the SSTR5 receptor modulator is a gut-restricted inverse agonist of a SSTR5 receptor.

26. The method of any one of the preceding claims, wherein the CCK_{A} receptor modulator is a gut-restricted agonist of a CCK_{A} receptor.

27. The method of any one of claims 20-26, wherein the gut-restricted agonist, gut-restricted antagonist, or gut-restricted inverse agonist comprises a covalently bonded kinetophore, optionally through a linker.

28. The method of any one of claims 20-26, wherein the at least two receptor modulators selected from the group consisting of a gut-restricted agonist, a gut-restricted antagonist, and a gut-restricted inverse agonist are covalently bonded, optionally through a linker.

29. The method of any one of claims 20-28, wherein the gut-restricted agonist, gut-restricted antagonist, or gut-restricted inverse agonist has <10% oral bioavailability, <8% oral bioavailability, <5% oral bioavailability, <3% oral bioavailability, or <2% oral bioavailability.

30. The method of any one of claims 20-29, wherein the unbound plasma levels of the gut-restricted agonist, gut-restricted antagonist, or gut-restricted inverse agonist are 10-fold, 20-fold, 50-fold, 100-fold, 500-fold, or 1000-fold lower than the IC₅₀ value or the EC₅₀ value of the gut-restricted agonist, gut-restricted antagonist, or gut-restricted inverse agonist against its receptor.

31. The method of any one of claims 1-30, wherein the condition involving the gut-brain axis is a metabolic disorder.

32. The method of claim 31, wherein the metabolic disorder is diabetes.

33. The method of claim 31, wherein the metabolic disorder is obesity.

34. The method of any one of claims 1-30, wherein the condition involving the gut-brain axis is a nutritional disorder.

35. The method of claim 34, wherein the nutritional disorder is short bowel syndrome, intestinal failure, or intestinal insufficiency.

36. The method of claim 34 or claim 35, wherein the nutritional disorder is short bowel syndrome

37. A pharmaceutical composition comprising at least two receptor modulators selected from the group consisting of a TGR5 receptor modulator, a GPR40 receptor modulator, a GPR119 receptor modulator, a somatostatin receptor 5 (SSTR5) modulator, and a CCK_{A} receptor modulator.

38. The pharmaceutical composition of claim 37, comprising:
i) a TGR5 receptor modulator and a SSTR5 receptor modulator;
ii) a TGR5 receptor modulator and a GPR40 receptor modulator;
iii) a TGR5 receptor modulator and a GPR119 receptor modulator;
iv) a TGR5 receptor modulator and a CCK_{A} receptor modulator;
v) a GPR40 receptor modulator and a GPR119 receptor modulator;
vi) a GPR40 receptor modulator and a SSTR5 receptor modulator;
vii) a GPR40 receptor modulator and a CCK_{A} receptor modulator;
viii) a GPR119 receptor modulator and a SSTR5 receptor modulator;
ix) a GPR119 receptor modulator and a CCK_{A} receptor modulator;
x) a SSTR5 receptor modulator and a CCK_{A} receptor modulator;
xi) a TGR5 receptor modulator, a SSTR5 receptor modulator, and a GPR119 receptor modulator;
xii) a TGR5 receptor modulator, a SSTR5 receptor modulator, and a GPR40 receptor modulator;
xiii) a TGR5 receptor modulator, a SSTR5 receptor modulator, and a CCK_{A} receptor modulator;
xiv) a TGR5 receptor modulator, a GPR40 receptor modulator, and a GPR119 receptor modulator;
xv) a TGR5 receptor modulator, a GPR40 receptor modulator, and a CCK_{A} receptor modulator;
xvi) a TGR5 receptor modulator, a GPR119 receptor modulator, and a CCK_{A} receptor modulator;
xvii) a GPR40 receptor modulator, a GPR119 receptor modulator, and a SSTR5 receptor modulator;
xviii) a SSTR5 receptor modulator, a GPR40 receptor modulator, and a CCK_{A} receptor modulator;
xix) a SSTR5 receptor modulator, a GPR119 receptor modulator, and a CCK_{A} receptor modulator;
xx) a GPR40 receptor modulator, a GPR119 receptor modulator, and a CCK_{A} receptor modulator;
xxi) a TGR5 receptor modulator, a GPR40 receptor modulator, a GPR119 receptor modulator, and a SSTR5 receptor modulator;
xxii) a TGR5 receptor modulator, a GPR40 receptor modulator, a GPR119 receptor modulator, and a CCK_{A} receptor modulator;
xxiii) a TGR5 receptor modulator, a GPR40 receptor modulator, a SSTR5 receptor modulator, and a CCK_{A} receptor modulator;
xxiv) a TGR5 receptor modulator, a GPR119 receptor modulator, a SSTR5 receptor modulator, and a CCK_{A} receptor modulator;
xxv) a GPR40 receptor modulator, a GPR119 receptor modulator, and a SSTR5 receptor modulator, and a CCK_{A} receptor modulator; or
xxvi) a TGR5 receptor modulator, a GPR40 receptor modulator, a GPR119 receptor modulator, a SSTR5 receptor modulator, and a CCK_{A} receptor modulator.

39. The pharmaceutical composition of claim 37, wherein the at least two receptor modulators are covalently bonded, optionally through a linker.
